(19) United States
(12) Patent Application Publication
(10) Pub. No.: US 2015/0126384 A1
(43) Pub. Date: May 7, 2015

(54) Prognostic Biomarkers in Patients with Ovarian Cancer

(71) Applicant: VERMILLION, INC., Austin, TX (US)

(72) Inventors: Estrid Hogdall, Copenhagen (DK); Eric T. Fung, Mountain View, CA (US); Jarle IB Christensen, Copenhagen (DK); Claus Hogdall, Copenhagen (DK)

(73) Assignee: VERMILLION, INC., Austin, TX (US)

(21) Appl. No.: 14/073,668

(22) Filed: Nov. 6, 2013

(51) Int. Cl. G01N 33/68

(52) U.S. Cl. CPC G01N 33/6893
USPC 506/9; 436/501; 435/7.4; 506/18

(57) ABSTRACT

The present invention provides methods for assessing an ovarian cancer patient's survival status. Also, methods for evaluating the ovarian cancer state of a patient are described herein. These methods involve the detection, analysis, and classification of biological patterns in biological samples. The biological patterns are obtained using, for example, mass spectrometry systems and other techniques.

### RELATED APPLICATIONS

This application claims the benefit of U.S. Provisional Application Ser. No. 61/406,044, filed Oct. 22, 2010 the entire contents of which are hereby incorporated herein by reference.

### BACKGROUND OF THE INVENTION

Ovarian cancer is among the most lethal gynecologic malignancies in developed countries. Annually in the United States alone, approximately 23,000 women are diagnosed with the disease and almost 14,000 women die from it. (Jamal, A., et al., CA Cancer J. Clin, 2002; 52:23-47). Despite progress in cancer therapy, ovarian cancer mortality has remained virtually unchanged over the past two decades. Given the steep survival gradient relative to the stage at which the disease is diagnosed, early detection remains the most important factor in improving long-term survival of ovarian cancer patients.

The poor prognosis of ovarian cancer diagnosed at late stages, the cost and risk associated with confirmatory diagnostic procedures, and its relatively low prevalence in the general population together pose extremely stringent requirements on the sensitivity and specificity of a test for it to be used for screening for ovarian cancer in the general population.

The identification of tumor markers suitable for the early detection and diagnosis of cancer holds great promise to improve the clinical outcome of patients. It is especially important for patients presenting with vague or no symptoms or with tumors that are relatively inaccessible to physical examination. Despite considerable effort directed at early detection, women generally present with disseminated disease at diagnosis.

Thus, there is a critical need to identify one or more panels of biomarkers that deliver the required sensitivity and specificity for early detection of ovarian cancer. Without an acceptable screening test, early detection remains the most critical factor in improving long-term survival of patients with ovarian cancer.

Although the stage of disease is one of the strongest predictors of survival in patients with ovarian cancer, disease stage alone is not adequate to predict survival or outcome in these patients. Improved methods for predicting a patient's prognosis could improve patient management by, for example, identifying patients in whom more aggressive therapy might be warranted or to whom personalized treatments might be offered.

Thus, it is desirable to have reliable and accurate methods for determining the ovarian cancer status of a subject, predicting overall survival of a subject or predicting progression free survival of a subject. The results of such methods are useful in managing subject treatment.

### SUMMARY

The present invention provides compositions and methods for determining ovarian cancer prognosis (e.g., predicting overall survival probability or predicting progression free survival probability). Such methods are useful in selecting an appropriate therapeutic regimen for the subject.

Advantageously, the invention provides compositions comprising one or more biomarkers and sensitive and rapid methods for using the kits to determine the survival status of patients with ovarian cancer by measuring the levels of particular biomarkers in a biological sample. The detection and measurement of these biomarkers in patient samples provides information that diagnosticians can correlate with survival status of human ovarian cancer patients or a negative diagnosis (e.g., normal or disease-free). In one embodiment, the markers are characterized by mass/charge ratio, molecular weight and/or by their known protein identities. The markers can be resolved from other proteins in a sample by using a variety of fractionation techniques, e.g., chromatographic separation coupled with mass spectrometry, protein capture using immobilized antibodies, bead-protein complexes or by traditional immunoassays. In preferred embodiments, the method of resolution involves Surface-Enhanced Laser Desorption/Ionization (“SELDI”) mass spectrometry or immunoassay.

In one aspect, the invention generally features a method of determining the prognosis of a subject having or suspected of having ovarian cancer, the method involving comparing the level of biomarkers inter-alpha (globulin) inhibitor H4 (plasma Kallikrein-sensitive glycoprotein), transferrin (TFR), and beta-2 microglobin (B2M) or fragments thereof in a sample from the subject to the level present in a reference, wherein an increased level of said biomarkers relative to the reference is indicative of a poor prognosis.

In another aspect, the invention generally features a method of determining the prognosis of a subject having or suspected of having ovarian cancer, the method involving comparing the level of biomarkers B2M, TrF and ITIH4 or fragments thereof, wherein an increased level of said biomarkers relative to the reference is indicative of a poor prognosis.

In another aspect, the invention generally features a method of determining the prognosis of a subject having or suspected of having ovarian cancer, the method involving comparing the level of biomarkers B2M and CTAP3 or fragments thereof, wherein an increased level of said biomarkers relative to the reference is indicative of a poor prognosis.

In one aspect, the invention generally features a method of determining the prognosis of a subject having or suspected of having ovarian cancer, the method involving comparing the level of biomarkers CA125, HEPC, B2M and CTAP3 or fragments thereof in a sample from the subject to the level present in a reference, wherein an increased level of said biomarkers relative to the reference is indicative of a poor prognosis.

In one aspect, the invention generally features a method of determining the prognosis of a subject having or suspected of having ovarian cancer, the method involving comparing the level of biomarkers APOA1, TT, HEPC, B2M, CTAP3, TrF and CA125 or fragments thereof in a sample from the subject to the level present in a reference, wherein an increased level of said biomarkers relative to the reference is indicative of a poor prognosis.

In one aspect, the invention generally features a method of qualifying ovarian cancer status in a human involving providing a subject sample of blood or a blood derivative; and fractionating proteins in the sample on an anion exchange resin and collecting fractions that contain inter-alpha (globulin) inhibitor H4 (plasma Kallikrein-sensitive glycoprotein) (ITIH4), transferrin (TFR), and beta-2 microglobin (B2M).

In one aspect, the invention generally features a kit containing a capture reagent that binds a panel of biomarkers containing, inter-alpha (globulin) inhibitor H4 (plasma Kallikrein-sensitive glycoprotein) (ITIH4), transferrin (TFR), and) beta-2 microglobin (B2M); and a container containing at the panel of biomarkers.

In one aspect, the invention generally features a kit containing capture reagents that binds the panel of biomarkers fragments containing inter-alpha (globulin) inhibitor H4 (plasma Kallikrein-sensitive glycoprotein) (ITIH4), transferrin (TFR), and) beta-2 microglobin (B2M); and instructions for using the capture reagents to detect the biomarkers.

In one aspect, the invention generally features a system containing a plurality of capture reagents each of which has bound to it a different biomarker containing inter-alpha (globulin) inhibitor H4 (plasma Kallikrein-sensitive glycoprotein) (ITIH4), transferrin (TFR), and) beta-2 microglobin (B2M).

In one aspect, the invention generally features a method of determining an ovarian cancer patient's prognosis containing determining the concentration or expression levels or peak intensity values of inter-alpha (globulin) inhibitor H4 (plasma Kallikrein-sensitive glycoprotein) (ITIH4), transferrin (TFR), and beta-2 microglobin (B2M); and correlating the measurements with ovarian cancer patient survival status.

In one aspect, the invention generally features a method of determining an ovarian cancer patient's prognosis involving determining the concentration or expression levels or peak intensity values of a combination of two or more biomarkers in a sample from the subject, wherein the one or more biomarkers are selected from the group consisting of: inter-alpha (globulin) inhibitor H4 (plasma Kallikrein-sensitive glycoprotein) (ITIH4), transferrin (TFR), and beta-2 microglobin (B2M) and correlating the measurements with ovarian cancer patient survival status.

In one aspect, the invention generally features a method of determining an ovarian cancer patient's prognosis involving determining the concentration or expression levels or peak intensity values of inter-alpha (globulin) inhibitor H4 (plasma Kallikrein-sensitive glycoprotein) (ITIH4), transferrin (TFR), and beta-2 microglobin (B2M); and correlating the measurements with ovarian cancer patient survival status.

In various embodiments of any of the above aspects or any other aspect of the invention delineated herein, the methods further involve comparing the level of one or more additional biomarkers to the level present in a reference, wherein the additional biomarkers are selected from the group consisting of apolipoprotein A1, transthyretin, inter-alpha trypsin inhibitor IV, transferrin, hepcidin, connective-tissue activating protein 3, and Serum Amyloid A1 and beta-2 microglobin. In other embodiments the methods further involve comparing the level of CA125 in the subject sample to the level present in a reference. In another embodiment the method further comprises considering one or more of the following: radicality of primary surgery, age at diagnosis and treatment. In other embodiments the method further comprises considering one or more of FIGO stage, histological type of tumor, and CA125. In yet other embodiments the prognosis is predictive of overall survival or progression-free survival. In further embodiments failure to detect an increased level in one or more of said biomarkers is indicative of a good prognosis. In yet other embodiments a patient's prognosis is used in selecting a therapeutic regiment. In further embodiments, a poor prognosis indicates that the subject requires an aggressive therapeutic regimen and a good prognosis indicates that the subject requires a less aggressive therapeutic regimen. In yet other embodiments an aggressive therapeutic regimen includes neoadjuvant chemotherapy.

In various embodiments of any of the above aspects or any other aspect of the invention delineated herein, the overall survival or progression free survival is selected from the group consisting of one to two years survival post diagnosis; two to five years post diagnosis; and beyond five years post diagnosis. In other embodiments the panel of biomarkers is measured by immunoassay, mass spectrometry, or radioassay. In additional embodiments the panel of biomarkers is captured using immobilized antibodies. In yet other embodiments the panel of biomarkers is detected using immobilized antibodies. In certain embodiments the correlating is performed by a software classification algorithm. In yet other embodiments the sample is selected from ovarian tissue, lymph nodes, tissue biopsy (e.g., diaophram, intestine, lavage, omentum) ovarian cyst fluid, ascites, pleural effusion, urine, blood, serum, and plasma.

In various embodiments of any of the above aspects or any other aspect of the invention delineated herein, the capture reagent is an antibody. In other embodiments contain an MS probe to which the capture reagents are attached or is attachable. In other embodiments the capture reagents are immobilized metal chelates. In yet other embodiments the kits contain written instructions for use of the kit for detection of ovarian cancer status in a subject.

In various embodiments of any of the above aspects or any other aspect of the invention delineated herein, an article of manufacture containing a panel of capture reagents that bind the panel of biomarkers or fragments of the respective biomarkers thereof. In yet other embodiments the biomarkers are inter-alpha (globulin) inhibitor H4 (plasma Kallikrein-sensitive glycoprotein) (ITIH4), transferrin (TFR), and beta-2 microglobin (B2M). In other embodiments the biomarkers are inter-alpha (globulin) inhibitor H4 (plasma Kallikrein-sensitive glycoprotein) (ITIH4), transferrin (TFR), and) beta-2 microglobin (B2M).

More specifically, it has been discovered that measuring particular combinations of biomarkers provides a surprisingly accurate prognosis for subjects having ovarian cancer. The panel of biomarkers consists of inter-alpha (globulin) inhibitor H4 (plasma Kallikrein-sensitive glycoprotein) (ITIH4), transferrin (TRF), and beta-2 microglobin (B2M). This panel of three biomarkers has been shown by the instant inventors to be highly indicative of the prognosis of subjects having ovarian cancer.

Moreover, the panel of biomarkers is predictive of survival independent of the stage of cancer.

The present invention provides a method of assessing an ovarian cancer patient's survival status in a subject containing (a) measuring the panel of three biomarkers in a sample from the subject, and correlating the measurement with ovarian cancer patient survival status. In certain methods, the measuring step comprises detecting the m/z (mass-to-charge ratio) values of markers in the sample using SELDI.

The instant invention provides methods for determining both progression free survival and overall survival in subjects diagnosed with ovarian cancer.

Preferred methods of the invention also include assessing ovarian cancer patient survival status comprising:

determining the concentration or expression levels of the panel of three biomarkers in a sample from the subject, wherein the three biomarkers are from the inter-alpha (globulin) inhibitor H4 (plasma Kallikrein-sensitive glycoprotein) (ITIH4), transferrin (TFR), and beta-2 microglobin (B2M), and correlating the corresponding concentration or expression levels with ovarian cancer patient survival status.

In certain embodiments, the methods further comprise managing subject treatment based on the status determined by the methods disclosed herein. For example, if the result of the methods of the present invention is inconclusive or there is reason that confirmation of status is necessary, the physician may order more tests. Alternatively, if the result of the methods of the present invention indicate a potentially poor prognosis, alternative or more aggressive therapies may be warranted. Furthermore, if the results show a potentially good prognosis, no or less aggressive therapies may be warranted.

Examples of more aggressive therapy include: a) The physician may after surgery treat the patient with more intensive and prolonged chemotherapy. b) Offer additional chemotherapy or biological treatments. c) The patient may be monitored more closely for relapse or progressive disease. d) Patients with both an indication of a poor prognosis and extensive disease, which on imaging indicate nonradical surgery, may be offered neoadjuvant chemotherapy and subsequent interval surgery. e) The proteomic index may be part in the total clinical judgment of treatment versus palliative treatment in severe ill patients. f) Radical and correct staged patients with stage one and grade 1-2 may be offered adjuvant treatment. g) The patients must be selected for surgery by a gynecologic-oncologic surgeon experienced in performing extensive procedures Examples of less aggressive therapy include, a) The index may be part in the decision making for radical surgery. b) Radical and correct staged patients with stage one and grade 1-2 may avoid a potentially harmful chemotherapy. c) The patient may be operated by a less specialized gynecologist.

A prognostic index may in the future be used to select patients for individualized new treatments (e.g. antibody or molecular based). This may be specially the case were some of the proteins or precursors are targets for the therapy

The term “ovarian cancer patient survival status” refers to the status of survival of the patient. Examples of types of ovarian cancer survival statuses include, but are not limited to, disease free or overall survival one year after diagnosis, 2 years after diagnosis, 3 years after diagnosis, 4 years after diagnosis, and 5 or more years after diagnosis. Another type of status is “treatment responsiveness” i.e. whether a patient has a high or low likelihood of responding to a given type of therapy. A third type of status is “remission” i.e. whether a patient is deemed to be free of disease (in remission) or to have cancer after one more therapeutic interventions (in recurrence). Other statuses and degrees of each status are known in the art.

For the mass values of the markers disclosed herein, the mass accuracy of the spectral instrument is considered to be about within +/−0.15 percent of the disclosed molecular weight value. Additionally, to such recognized accuracy variations of the instrument, the spectral mass determination can vary within resolution limits of from about 400 to 1000 m/dm, where m is mass and dm is the mass spectral peak width at 0.5 peak height. Those mass accuracy and resolution variances associated with the mass spectral instrument and operation thereof are reflected in the use of the term “about” in the disclosure of the mass of each of seven biomarkers. It is also intended that such mass accuracy and resolution variances and thus meaning of the term “about” with respect to the mass of each of the markers disclosed herein is inclusive of variants of the markers as may exist due to genotype and/or ethnicity of the subject and the particular cancer or origin or stage thereof.

A Cox proportional hazards model is a regression model for studying the association between time to event data and explanatory variables, e.g. tumor stage, age and gender. The hazard rate (intensity of the event) on the log scale is the dependent variable which is a linear function of the explanatory variables. The effect is presented by the hazard ratio similar to the relative risk concept. A HR above one indicates an increased intensity or risk for the event and a value below a decreased intensity or risk. For example, in our study is HR=1.62 for a patient with a stage III ovarian cancer compared to a patient with a stage I cancer. This means that the stage III patient has an increased intensity or risk of 62% for death compared to the stage I patient. In the analysis it is also found, that a patient in the highest level of our proteomic index has a RH=2.64, corresponding to an increased intensity or risk of death of 164% compared to a patient with proteomic index one unit lower. Corresponding to this indicates a HR above one a poor prognosis and a HR below one a more favorable prognosis.

A statistical test specifies a null hypothesis which is compared to the alternative hypothesis based on the probability of the observed outcome. If the probability of observing the outcome assuming the null hypothesis is below a prespecified threshold denoted the level of significance then the null hypothesis is rejected in favor of the alternative hypothesis. The probability of incorrectly rejecting the null hypothesis, i.e. the null hypothesis is true, is the chosen level of significance often denoted the Type I error. A good result is the rejection of the null hypothesis when the alternative is true, the probability of this is called the power of the test and is dependent on the difference compared to the null hypothesis and the chosen level of significance.

Methods of measuring the biomarkers include use of a biochip array. Biochip arrays useful in the invention include protein and nucleic acid arrays. One or more markers are captured on the biochip array and subjected to laser ionization to detect the molecular weight of the markers. Analysis of the markers is, for example, by molecular weight of the one or more markers against a threshold intensity that is normalized against total ion current. Preferably, logarithmic transformation is used for reducing peak intensity ranges to limit the number of markers detected.

Another method of measuring the biomarkers includes the use of a combinatorial ligand library synthesized on beads as described in U.S. Ser. No. 11/495,842, filed Jul. 28, 2006 and entitled “Methods for Reducing the range in Concentrations of Analyte Species in a Sample”; hereby incorporated by reference in its entirety.

In other methods of the present invention, the step of correlating the measurement of the biomarkers with ovarian cancer patient survival status is performed by a software classification algorithm. For example, data is generated on subject samples on a biochip array, by subjecting said biochip array to laser ionization and detecting intensity of signal for mass/charge ratio; and, transforming the data into computer readable form; and executing an algorithm that classifies the data according to user input parameters, for detecting signals that represent markers present in ovarian cancer patients and are lacking in non-cancer subject controls.

Biochip surfaces are, for example, ionic, anionic, comprised of immobilized nickel ions, comprised of a mixture of positive and negative ions, comprised of one or more antibodies, single or double stranded nucleic acids, proteins, peptides or fragments thereof, amino acid probes, or phage display libraries.

In other preferred methods one or more of the markers are measured using laser desorption/ionization mass spectrometry, comprising providing a probe adapted for use with a mass spectrometer comprising an adsorbent attached thereto, and contacting the subject sample with the adsorbent, and; desorbing and ionizing the marker or markers from the probe and detecting the deionized/ionized markers with the mass spectrometer.

Preferably, the laser desorption/ionization mass spectrometry comprises: providing a substrate comprising an adsorbent attached thereto; contacting the subject sample with the adsorbent; placing the substrate on a probe adapted for use with a mass spectrometer comprising an adsorbent attached thereto; and, desorbing and ionizing the marker or markers from the probe and detecting the desorbed/ionized marker or markers with the mass spectrometer.

The adsorbent can for example be hydrophobic, hydrophilic, ionic or metal chelate adsorbent, such as, nickel or an antibody, single- or double stranded oligonucleotide, amino acid, protein, peptide or fragments thereof.

The methods of the present invention can be performed on any type of patient sample that would be amenable to such methods, e.g., blood, serum and plasma.

The present invention also provides kits comprising capture reagents that bind the biomarkers and a container comprising the panel of biomarkers. While the capture reagent can be any type of reagent, preferably the reagent is a SELDI probe.

In certain kits of the present invention, the capture reagent comprises an immobilized metal chelate (“IMAC”).

Certain kits of the present invention further comprise a wash solution that selectively allows retention of the bound biomarker to the capture reagent as compared with other biomarkers after washing.

The invention also provides kits comprising capture reagents that bind the three biomarkers and instructions for using the capture reagent to measure the biomarkers. In certain of these kits, the capture reagent comprises an antibody. Furthermore, some kits further comprise an MS probe to which the capture reagent is attached or is attachable. In some kits, the capture reagent comprises an IMAC. The kits may also contain a wash solution that selectively allows retention of the bound biomarker to the capture reagent as compared with other biomarkers after washing. Preferably, the kit comprises written instructions for use of the kit for determining ovarian cancer status and the instructions provide for contacting a test sample with the capture reagents and measuring one or more biomarkers retained by the capture reagents.

The kit also provides for capture reagents, which are antibodies, single or double stranded oligonucleotide, amino acid, protein, peptide or fragments thereof.

Measurement of one or more protein biomarkers using the kit, is by mass spectrometry or immunoassays such as an ELISA.

Purified proteins for detection of ovarian cancer and/or generation of antibodies for further diagnostic assays are also provided for.

The invention also provides an article manufacture comprising capture reagents bound to the panel of biomarkers.

Other aspects of the invention are described infra.

### BRIEF DESCRIPTION OF THE DRAWINGS

FIGS. 1A-1D depicts representative spectra from non-progressing OC patients (top two spectra) and progressing OC patients (bottom two spectra). A, transthyretin (TRF); B, beta 2 microglobulin (B2M); C, ITIH4; D, CTAP3.

FIGS. 2A-2B depicts Kaplan-Meier curves describing the association between the xb-pro index and A. patients with residual tumor after surgery (N=92) and B. all ovarian cancer patients (N=150). Patients were divided into three groups using the first and second tertiles of the xb-pro index as cutpoints. For both patient groups a highly significant better survival was observed between patients with xb-pro index in the upper tertile compared with patients with lower xb-pro index values.

FIG. 3 depicts a plot showing hazard ratios for different combinations of the 3 intensities, B2M on the abscissae and for 1 and third quartiles of TRF and ITIH4, all HR compared to a patient with a median level of each peak.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Adsorption” refers to detectable non-covalent binding of an analyte to an adsorbent or capture reagent.

“Analyte” refers to any component of a sample that is desired to be detected. The term can refer to a single component or a plurality of components in the sample.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface and, therefore, function as probes.

The “complexity” of a sample adsorbed to an adsorption surface of an affinity capture probe means the number of different protein species that are adsorbed.

The phrase “differentially present” refers to differences in the quantity and/or the frequency of a marker present in a sample taken from a subject having or having a propensity to develop cancer as compared to a control subject. For example, the IAIH4 fragment is present at an elevated level in biological samples obtained from ovarian cancer patients as compared to samples from control subjects. In contrast, Apo A1 and transthyretin described herein are present at a decreased level in samples obtained from ovarian cancer patients compared to samples from control subjects. Furthermore, a marker can be a polypeptide, which is detected at a higher frequency or at a lower frequency in samples of human cancer patients compared to samples of control subjects. A marker can be differentially present in terms of level, quantity, and/or frequency.

A polypeptide is differentially present between two samples if the amount/level of the polypeptide in one sample is different from the amount of the polypeptide in the other sample. Preferably, the difference is statistically significant. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between two sets of samples if the frequency of detecting the polypeptide in the ovarian cancer patients' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition, i.e., ovarian cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “control amount” of a marker can be any amount or a range of amount, which is to be compared against a test amount of a marker. For example, a control amount of a marker can be the amount of a marker in a person without ovarian cancer. In one embodiment, a control amount is an absolute amount (e.g., μg/ml). In another embodiment, a control amount is the relative level (e.g., relative intensity of signals).

A “diagnostic amount” of a marker refers to an amount of a marker in a subject's sample that is consistent with a diagnosis of ovarian cancer. In one embodiment, a diagnostic amount is the absolute amount (e.g., μg/ml) of analyte. In another embodiment, a diagnostic amount is the relative level (e.g., relative intensity of signals).

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionuclides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Laser desorption mass spectrometer” refers to a mass spectrometer that uses laser energy as a means to desorb, volatilize, and ionize an analyte.

“Managing subject treatment” refers to the action of a clinician (e.g., physician (subsequent to a determination of ovarian cancer status in a subject. For example, if the result of the methods of the present invention is inconclusive or there is reason that confirmation of status is necessary, the physician may order more tests. Alternatively, if the result of the methods of the present invention indicates a potentially poor prognosis, alternative or more aggressive therapies may be warranted. Furthermore, if the results show a potentially good prognosis, no or less aggressive therapies may be warranted.

Examples of more aggressive therapy include: a) The physician may after surgery treat the patient with more intensive and prolonged chemotherapy. b) Offer additional chemotherapy or biological treatments. c) The patient may be monitored more closely for relapse or progressive disease. d) Patients with both an indication of a poor prognosis and extensive disease, which on imaging indicate nonradical surgery, may be offered neoadjuvant chemotherapy and subsequent interval surgery. e) The proteomic index may be part in the total clinical judgment of treatment versus palliative treatment in severe ill patients. f) Radical and correct staged patients with stage one and grade 1-2 may be offered adjuvant treatment. g) The patients may be selected for surgery by a gynecologic-oncologic surgeon experienced in performing extensive procedures. Examples of less aggressive therapy include. a) The index may be part of the decision making for radical surgery. b) Radical and correct staged patients with stage one and grade 1-2 may avoid a potentially harmful chemotherapy. c) The patient may be operated on by a less specialized gynecologist.

A prognostic index may in the future be used to select patients for individualized treatment (e.g. antibody or molecular based). In one embodiment, a protein of the invention is the targets of the therapy.

“Marker” in the context of the present invention refers to a polypeptide that is differentially present in a sample taken from a patients having human cancer as compared to a reference. In one embodiment, the reference is a comparable sample taken from a control subject. A control subject may be a person with a negative diagnosis or undetectable cancer, such as a normal or healthy subject. The term “biomarker” is used interchangeably with the term “marker.”

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to SELDI and immunoassay. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

“Mass analyzer” refers to a sub-assembly of a mass spectrometer that comprises a means for measuring a parameter that can be translated into mass-to-charge ratios of gas phase ions. In a time-of-flight mass spectrometer the mass analyzer comprises an ion optic assembly, a flight tube and an ion detector.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, and combinations thereof.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Solid support” refers to a solid material which can be derivatized with, or otherwise attached to, a capture reagent. Exemplary solid supports include probes, microtiter plates and chromatographic resins.

“Three biomarker panel” refers to a set of biomarkers identified herein. In one embodiment, the three biomarkers are inter-alpha (globulin) inhibitor H4 (plasma Kallikrein-sensitive glycoprotein) (ITIH4), transferrin (TFR), and beta-2 microglobin (B2M).

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, WCX-2, CM-10, IMAC-3, MAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 biochip has quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu++ and Ni++, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application US20030032043A1 (Pohl and Papanu, “Latex Based Adsorbent Chip,” Jul. 16, 2002) and U.S. patent application 60/350,110 (Urn et al., “Hydrophobic Surface Chip,” Nov. 8, 2001).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

A “test amount” of a marker refers to an amount of a marker present in a sample being tested. A test amount can be either in absolute amount (e.g., μg/ml) or a relative amount (e.g., relative intensity of signals).

### DETAILED DESCRIPTION OF THE INVENTION

The present invention provides compositions and methods for determining the prognosis of subjects having or suspected of having ovarian cancer by detecting particular biomarkers. The detection and measurement of these biomarkers in subject samples provides information that diagnosticians can correlate with overall survival and/or progression-free survival to select an appropriate therapeutic regimen for the subject.

The invention is based, at least in part, on the discovery that one or more of the following biomarkers are useful for detecting and/or characterizing ovarian cancer in a subject: apolipoprotein A1 (APOA1), transthyretin (cysteinylated form) (TT), inter-alpha trypsin inhibitor IV (internal fragment) (ITIH4), transferrin (TrF), hepcidin (HEPC), connective-tissue activating protein 3 (CTAP3), Serum Amyloid A1 (SAA), and beta-2 microglobin (B2M). In particular embodiments, these biomarkers are used to determine a subject's prognosis (e.g., likely overall survival and/or progression free survival). In particular embodiments, the biomarkers used are inter-alpha (globulin) inhibitor H4 (plasma Kallikrein-sensitive glycoprotein) (ITIH4), transferrin (TFR), and/or beta-2 microglobin (B2M).

These biomarkers have been disclosed in PCT/US2005/010783 (WO 2005/098447); US Patent Application Publication 2005/0059013; PCT/US03/00531 (WO03/057014); PCT/US2003/024636 (WO 2004/012588); and PCT/US06/08578, all of which documents are incorporated herein by reference in their entirety.

These biomarkers assess a patient's survival status after having developed ovarian cancer and could potentially provide additional information to physicians for clinical decision-making. This is supported by Cox multivariate analysis in an independent validation. For example, several large-scale studies have suggested that ovarian cancer patients with surgical procedures operated by gynecological oncologists tend to have a better long-term survival. However, other studies concluded that currently only about one third of ovarian cancer patients undergoing surgical procedures in the US are treated by gynecological oncologists. With the current total number of gynecological oncologists available, it is still not practical to have all patients undergoing surgery for suspected ovarian cancer be operated by gynecologic oncologists. The biomarkers have the potential to be used to identify patients with the lower probability of surviving ovarian cancer and recommend them for treatment by gynecologic oncologists.

High-throughput protein profiling combined with effective use of bioinformatics tools provides a useful approach to screening for cancer markers. Briefly, the system used in the present invention utilizes chromatographic ProteinChip® Arrays to assay samples using SELDI (Surface Enhanced Laser Desorption/Ionization). Proteins bound to the arrays are read in a ProteinChip® Reader, a time-of-flight mass spectrometer.

The present invention is based upon the discovery of protein markers that are differentially present in samples of ovarian cancer patients and control subjects, and the application of this discovery in methods and kits for determining ovarian cancer status. These protein markers are found in samples from ovarian cancer patients at levels that are different than the levels in samples from women in whom human cancer is undetectable. Accordingly, the amount of one or more markers found in a test sample compared to a control, or the presence or absence of one or more markers in the test sample provides useful information regarding the ovarian cancer status of the patient.

Due to the dismal prognosis of late stage ovarian cancer, it is the general consensus that a physician will accept a test with a minimal positive predictive value of 10%. Extending this to the general population, a general screening test would require a sensitivity greater than 70% and a specificity of 99.6%. Currently, none of the existing serologic markers, such as CA125, CA72-4, or M-CSF, individually delivers such a performance. (Bast, R. C., et al., Int J Biol Markers, 1998; 13:179-87).

The best-characterized tumor marker, CA125, is negative in approximately 30-40% of stage I ovarian carcinomas and its levels are elevated in a variety of benign diseases. Its use as a population-based screening tool for early detection and diagnosis of ovarian cancer is hindered by its low sensitivity and specificity. Although pelvic and more recently vaginal sonography has been used to screen high-risk patients, neither technique has sufficient sensitivity and/or specificity to be applied to the general population. Recent efforts in using CA125 in combination with additional tumor markers (Woolas R P X F, et al., J Natl Cancer Inst, 1993; 85(21):1748-51; Woolas R P, et al., Gynecol Oncol, 1995; 59(1):111-6; Zhang Z, et al., Gynecol Oncol, 1999; 73(1):56-61; Zhang Z, et al., Use of Multiple Markers to Detect Stage I Epithelial Ovarian Cancers: Neural Network Analysis Improves Performance. American Society of Clinical Oncology 2001; Annual Meeting, Abstract) in a longitudinal risk of cancer model (Skates S J, et al., Cancer, 1995; 76(10 Suppl):2004-10), and in tandem with ultrasound as a second line test (Jacobs I D A, et al., Br Med J, 1993; 306(6884):1030-34; Menon U T A, et al., British Journal of Obstetrics and Gynecology, 2000; 107(2):165-69) have shown promising results in improving overall test specificity, which is critical for a disease such as ovarian cancer that has a relatively low prevalence.

Description of the Biomarkers

ITIH4 Fragments

Other biomarkers that are useful in the methods of the present invention one or more of a closely related set of cleavage fragments of inter-α-trypsin inhibitor heavy chain H4 precursor, also referred to alternatively herein as “ITIH4 fragments.” ITIH4 fragments are described as biomarkers for ovarian cancer in US patent publication 2005-0059013 A1, International Patent Publication WO 2005/098447 and Fung et al., Int. J. Cancer 115:783-789 (2005). ITIH4 fragments can be selected from the group consisting of ITIH4 fragment no. 1, ITIH4 fragment no. 2, and ITIH4 fragment no. 3.

The amino acid sequences of the ITIH4 fragments were determined to be: ITIH4 fragment 1 (SEQ ID NO: 5): MNFRPGVLSSRQLGLPGPPDVPDHAAYHPF ITIH4 fragment 2 (SEQ ID NO: 6): PGVLSSRQLGLPGPPDVPDHAAYHPF ITIH4 fragment 3 (SEQ ID NO: 7): GVLSSRQLGLPGPPDVPDHAAYHPF. The present invention also includes all other known fragments of ITIHA4.

ITIH4 precursor is a 930 amino acid protein (SwissProt Q14624). ITIH4 fragment 1 spans amino acids 658-687 of human ITIH4 precursor. ITIH4 fragment 2 spans amino acids 662-687 of ITIH4 precursor. ITIH4 fragment 3 spans amino acids 663-687 of ITIH4 precursor.

Additionally, preferred methods of the present invention include the use of modified forms of ITIH4 fragment. Modification of ITIH4 fragment may include the post-translational addition of various chemical groups, for example, glycosylation, lipidation, cysteinylation, and glutathionylation.

Transferrin (TRF)

Another biomarker that is useful in the methods of the present invention is transferrrin. Transferrrin is described as a biomarker for ovarian cancer in US patent publication 2005-0214760 A1. Transferrrin is a 679 amino acid protein derived from a 698 amino acid precursor (GenBank Accession No. NP_{—}001054 GI:4557871; SwissProt Accession No. P02787) (SEQ ID NO: 10). Transferrrin is recognized by antibodies available from, e.g., Dako (catalog A006) (www.dako.com, Glostrup, Denmark). Transferrin is glycosylated. Therefore, the measured molecular weight is higher than the theoretical weight, which does not take glycosylation into account.

Beta-2 Microglobin (B2M)

Another biomarker that is useful in the methods of the present invention is β2-microglobulin. β2-microglobulin is described as a biomarker for ovarian cancer in US provisional patent publication 60/693,679, filed Jun. 24, 2005 (Fung et al.). β2-microglobulin is a 99 amino acid protein derived from an 119 amino acid precursor (GI:179318; SwissProt Accession No. P61769) (SEQ ID NO: 11). β2-microglobulin is recognized by antibodies available from, e.g., Abcam (catalog AB759) (www.abcam.com, Cambridge, Mass.).

Because, in one embodiment, the biomarkers of this invention are characterized by mass-to-charge ratio, binding properties and spectral shape, they can be detected by mass spectrometry without knowing their specific identity. However, if desired, biomarkers whose identity is not determined can be identified by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes. Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

U.S. patent application Ser. No. 11/373,833, filed Mar. 10, 2006 is hereby incorporated by reference in its entirety.

It has been found that proteins frequently exist in a sample in a plurality of different forms characterized by a detectably different mass. These forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Modified forms of the biomarker of this invention also may be used, themselves, as biomarkers. In certain cases the modified forms may exhibit better discriminatory power in diagnosis than the specific forms set forth herein.

Modified forms of a biomarker can be initially detected by any methodology that can detect and distinguish the modified from the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, aptamers or Affibodies that recognize the biomarker and modified forms of it. This method also will also result in the capture of protein interactors that are bound to the proteins or that are otherwise recognized by antibodies and that, themselves, can be biomarkers. In certain embodiments, the biospecific capture reagents are bound to a solid phase. Then, the captured proteins can be detected by SELDI mass spectrometry or by eluting the proteins from the capture reagent and detecting the eluted proteins by traditional MALDI or by SELDI. The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

In yet another embodiment, the surfaces of biochips can be derivatized with the capture reagents directed against protein clusters either in the same location or in physically different addressable locations. One advantage of capturing different clusters in different addressable locations is that the analysis becomes simpler.

After identification of modified forms of a protein and correlation with the clinical parameter of interest, the modified form can be used as a biomarker in any of the methods of this invention. At this point, detection of the modified form can be accomplished by any specific detection methodology including affinity capture followed by mass spectrometry, or traditional immunoassay directed specifically the modified form. Immunoassay requires biospecific capture reagents, such as antibodies, to capture the analytes. Furthermore, if the assay must be designed to specifically distinguish protein and modified forms of protein. This can be done, for example, by employing a sandwich assay in which one antibody captures more than one form and second, distinctly labeled antibodies, specifically bind, and provide distinct detection of, the various forms. Antibodies can be produced by immunizing animals with the biomolecules. This invention contemplates traditional immunoassays including, for example, sandwich immunoassays including ELISA or fluorescence-based immunoassays, as well as other enzyme immunoassays.

### II. Test Samples

A) Subject Types

Samples are collected from women who have been diagnosed with ovarian cancer in whom the test is being used to determine their prognosis. Samples may be collected from women who had been diagnosed with ovarian cancer and received treatment to eliminate the cancer, or perhaps are in remission. In a preferred embodiment, the subjects are women who have been previously diagnosed as having ovarian cancer.

B) Types of Sample and Preparation of the Sample

The markers can be measured in different types of biological samples. The sample is preferably a biological fluid sample. Examples of a biological fluid sample useful in this invention include blood, blood serum, plasma, vaginal secretions, urine, ovarian cyst fluid, tears, saliva, etc. Because all of the markers are found in blood serum, blood serum is a preferred sample source for embodiments of the invention.

If desired, the sample can be prepared to enhance detectability of the markers. For example, to increase the detectability of markers, a blood serum sample from the subject can be preferably fractionated by, e.g., Cibacron blue agarose chromatography and single stranded DNA affinity chromatography, anion exchange chromatography, affinity chromatography (e.g., with antibodies) and the like. The method of fractionation depends on the type of detection method used. Any method that enriches for the protein of interest can be used. Sample preparations, such as pre-fractionation protocols, are optional and may not be necessary to enhance detectability of markers depending on the methods of detection used. For example, sample preparation may be unnecessary if antibodies that specifically bind markers are used to detect the presence of markers in a sample.

Typically, sample preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis. Examples of methods of fractionation are described in PCT/US03/00531 (incorporated herein in its entirety).

Preferably, the sample is pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomolecules in a sample that are more negatively charged from other types of biomolecules. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In preferred embodiments, the serum samples are fractionated via anion exchange chromatography. Signal suppression of lower abundance proteins by high abundance proteins presents a significant challenge to SELDI mass spectrometry. Fractionation of a sample reduces the complexity of the constituents of each fraction. This method can also be used to attempt to isolate high abundance proteins into a fraction, and thereby reduce its signal suppression effect on lower abundance proteins. Anion exchange fractionation separates proteins by their isoelectric point (pI). Proteins are comprised of amino acids, which are ambivalent-their charge changes based on the pH of the environment to which they are exposed. A protein's pI is the pH at which the protein has no net charge. A protein assumes a neutral charge when the pH of the environment is equivalent to pI of the protein. When the pH rises above the pI of the protein, the protein assumes a net negative charge. Similarly, when the pH of the environment falls below the pI of the protein, the protein has a net positive charge. The serum samples were fractionated according to the protocol set forth in the Examples below to obtain the markers described herein.

After capture on anion exchange, proteins were eluted in a series of step washes at pH 9, pH 7, pH 5, pH 4 and pH 3. A panel of three potential biomarkers was discovered by UMSA analysis of profiling data of three fractions (pH 9/flow through, pH 4, and organic solvent). Two of the peaks were from fraction pH 4 at m/z of 12828 and 28043, both down-regulated in the cancer group, and the third was from fraction pH 9/flow through at m/z of 3272, up-regulated in the cancer group. All bound to the immobilized metal affinity chromatography array charged with copper ions (IMAC3-Cu).

Biomolecules in a sample can also be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomolecules, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomolecules in a sample are separated by, e.g., isoelectric focusing, during which biomolecules in a sample are separated in a pH gradient until they reach a spot where their net charge is zero (i.e., isoelectric point). This first separation step results in one-dimensional array of biomolecules. The biomolecules in one-dimensional array is further separated using a technique generally distinct from that used in the first separation step. For example, in the second dimension, biomolecules separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomolecules. Typically, two-dimensional gel electrophoresis can separate chemically different biomolecules in the molecular mass range from 1000-200,000 Da within complex mixtures. The pI range of these gels is about 3-10 (wide range gels).

Biomolecules in the two-dimensional array can be detected using any suitable methods known in the art. For example, biomolecules in a gel can be labeled or stained (e.g., Coomassie Blue or silver staining). If gel electrophoresis generates spots that correspond to the molecular weight of one or more markers of the invention, the spot can be further analyzed by gas phase ion spectrometry. For example, spots can be excised from the gel and analyzed by gas phase ion spectrometry. Alternatively, the gel containing biomolecules can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI (e.g., using ProteinChip® array) as described herein.

Prior to gas phase ion spectrometry analysis, it may be desirable to cleave biomolecules in the spot into smaller fragments using cleaving reagents, such as proteases (e.g., trypsin). The digestion of biomolecules into small fragments provides a mass fingerprint of the biomolecules in the spot, which can be used to determine the identity of markers if desired.

High performance liquid chromatography (HPLC) can also be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers. For example, the spots can be analyzed using either MALDI or SELDI (e.g., using ProteinChip® array) as described herein.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomolecules can be modified to improve detection resolution. For instance, neuraminidase can be used to remove terminal sialic acid residues from glycoproteins to improve binding to an anionic adsorbent (e.g., cationic exchange ProteinChip® arrays) and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

### III. Capture of Markers

Biomarkers can be captured with capture reagents immobilized to a solid support, such as any biochip described herein, a multiwell microtiter plate or a resin. In particular, the biomarkers of this invention are preferably captured on SELDI protein biochips. Capture can be on a chromatographic surface or a biospecific surface. Any of the SELDI protein biochips comprising reactive surfaces can be used to capture and detect the biomarkers of this invention. However, the biomarkers of this invention bind well to immobilized metal chelates. The IMAC-3 and IMAC 30 biochips, which nitriloacetic acid functionalities that adsorb transition metal ions, such as Cu⁺⁺ and Ni⁺⁺, by chelation, are the preferred SELDI biochips for capturing the biomarkers of this invention. Any of the SELDI protein biochips comprising reactive surfaces can be used to capture and detect the biomarkers of this invention. These biochips can be derivatized with the antibodies that specifically capture the biomarkers, or they can be derivatized with capture reagents, such as protein A or protein G that bind immunoglobulins. Then the biomarkers can be captured in solution using specific antibodies and the captured markers isolated on chip through the capture reagent.

In general, a sample containing the biomarkers, such as serum, is placed on the active surface of a biochip for a sufficient time to allow binding. Then, unbound molecules are washed from the surface using a suitable eluant, such as phosphate buffered saline. In general, the more stringent the eluant, the more tightly the proteins must be bound to be retained after the wash. The retained protein biomarkers now can be detected by appropriate means.

### IV. Detection and Measurement of Markers

Once captured on a substrate, e.g., biochip or antibody, any suitable method can be used to measure a marker or markers in a sample. For example, markers can be detected and/or measured by a variety of detection methods including for example, gas phase ion spectrometry methods, optical methods, electrochemical methods, atomic force microscopy and radio frequency methods. Using these methods, one or more markers can be detected.

A) SELDI

One preferred method of detection and/or measurement of the biomarkers uses mass spectrometry and, in particular, “Surface-enhanced laser desorption/ionization” or “SELDI”. SELDI refers to a method of desorption/ionization gas phase ion spectrometry (e.g., mass spectrometry) in which the analyte is captured on the surface of a SELDI probe that engages the probe interface. In “SELDI MS,” the gas phase ion spectrometer is a mass spectrometer. SELDI technology is described in more detail above.

B) Immunoassay

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

An immunoassay is an assay that uses an antibody to specifically bind an antigen (e.g., a marker). The immunoassay is characterized by the use of specific binding properties of a particular antibody to isolate, target, and/or quantify the antigen. The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a marker from specific species such as rat, mouse, or human can be selected to obtain only those polyclonal antibodies that are specifically immunoreactive with that marker and not with other proteins, except for polymorphic variants and alleles of the marker. This selection may be achieved by subtracting out antibodies that cross-react with the marker molecules from other species.

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Typically a specific or selective reaction will be at least twice background signal or noise and more typically more than 10 to 100 times background.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of, or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art. Useful assays include, for example, an enzyme immune assay (ETA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Ten, eds., 7th ed. 1991); and Harlow & Lane, supra.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

C) Combinatorial Ligand Library Beads

Another method of measuring the biomarkers includes the use of a combinatorial ligand library synthesized on beads as described in U.S. Ser. No. 11/495,842, filed Jul. 28, 2006 and entitled “Methods for Reducing the range in Concentrations of Analyte Species in a Sample”; hereby incorporated by reference in its entirety.

### V. Data Analysis

When the sample is measured and data is generated the data is then analyzed by a computer software program. Generally, the software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human cancer and determines the closeness of fit between the two signals. The software also can include code indicating which the test sample is closest to, thereby providing a probable diagnosis.

In preferred methods of the present invention, multiple biomarkers are measured. The use of multiple biomarkers increases the predictive value of the test and provides greater utility in diagnosis, toxicology, patient stratification and patient monitoring. The process called “Pattern recognition” detects the patterns formed by multiple biomarkers greatly improves the sensitivity and specificity of clinical proteomics for predictive medicine. Subtle variations in data from clinical samples, e.g., obtained using SELDI, indicate that certain patterns of protein expression can predict phenotypes such as the presence or absence of a certain disease, a particular stage of cancer progression, or a positive or adverse response to drug treatments.

Data generation in mass spectrometry begins with the detection of ions by an ion detector as described above. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

In one example, Ciphergen's Biomarker Patterns™ Software is used to detect a pattern in the spectra that are generated. The data is classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

The spectra that are generated in embodiments of the invention can be classified using a pattern recognition process that uses a classification model. In some embodiments, data derived from the spectra (e.g., mass spectra or time-of-flight spectra) that are generated using samples such as “known samples” can then be used to “train” a classification model. A “known sample” is a sample that is pre-classified (e.g., cancer or not cancer). Data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” in any suitable manner. For example, signals above a predetermined signal-to-noise ratio can be selected so that a subset of peaks in a spectrum is selected, rather than selecting all peaks in a spectrum. In another example, a predetermined number of peak “clusters” at a common value (e.g., a particular time-of-flight value or mass-to-charge ratio value) can be used to select peaks. Illustratively, if a peak at a given mass-to-charge ratio is in less than 50% of the mass spectra in a group of mass spectra, then the peak at that mass-to-charge ratio can be omitted from the training data set. Pre-processing steps such as these can be used to reduce the amount of data that is used to train the classification model.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART-classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002/0193950 A1 (Gavin et al., “Method or analyzing mass spectra,” Dec. 19, 2002); U.S. 2003/0004402 A1 (Hitt et al., “Process for discriminating between biological states based on hidden patterns from biological data,” Jan. 2, 2003); and U.S. Pat. No. 7,113,896 A1 (Zhang and Zhang, “Systems and methods for processing biological expression data” Mar. 20, 2003).

More specifically, to obtain the biomarkers, the peak intensity data of samples from cancer patients and healthy controls were used as a “discovery set.” This data were combined and randomly divided into a training set and a test set to construct and test multivariate predictive models.

Generally, the data generated from Section IV above is inputted into a diagnostic algorithm (i.e., classification algorithm as described above). The classification algorithm is then generated based on the learning algorithm. The process involves developing an algorithm that can generate the classification algorithm. The methods of the present invention generate a more accurate classification algorithm by accessing a number of ovarian cancer and normal samples of a sufficient number based on statistical sample calculations. The samples are used as a training set of data on learning algorithm.

The generation of the classification, i.e., diagnostic, algorithm is dependent upon the assay protocol used to analyze samples and generate the data obtained in Section IV above. It is imperative that the protocol for the detection and/or measurement of the markers (e.g., in step IV) must be the same as that used to obtain the data used for developing the classification algorithm. The assay conditions, which must be maintained throughout the training and classification systems include chip type and mass spectrometer parameters, as well as general protocols for sample preparation and testing. If the protocol for the detection and/or measurement of the markers (step IV) is changed, the learning algorithm and classification algorithm must also change. Similarly, if the learning algorithm and classification algorithm change, then the protocol for the detection and/or measurement of markers (step IV) must also change to be consistent with that used to generate classification algorithm. Development of a new classification model would require accessing a sufficient number of ovarian cancer and normal samples, developing a new training set of data based on a new detection protocol, generating a new classification algorithm using the data and finally, verifying the classification algorithm with a multi-site study.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer. If it is separate from the mass spectrometer, the data must be inputted into the computer by some other means, whether manually or automated.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

### VI. Various Embodiments

In one embodiment, a serum sample is collected from a patient and then fractionated using an anion exchange resin as described above. In one embodiment, the biomarkers in the sample are captured using an IMAC copper ProteinChip array. The markers can then be detected using SELDI. In such a test one can detect inter-alpha (globulin) inhibitor H4 (plasma Kallikrein-sensitive glycoprotein) (ITIH4), transferrin (TFR), and beta-2 microglobin (B2M). The results are then entered into a computer system, which contains an algorithm that is designed using the same parameters that were used in the learning algorithm and classification algorithm to originally determine the biomarkers. The algorithm produces a diagnosis based upon the data received relating to each biomarker. For example, the algorithm can determine the chances of progression free survival (PFS) or overall survival (OS).

For example, the diagnosis is determined by examining the data produced from the SELDI tests with the classification algorithm that is developed using the biomarkers. The classification algorithm depends on the particulars of the test protocol used to detect the biomarkers. These particulars include, for example, sample preparation, chip type, mass spectrometer parameters and/or immunoassay conditions. If the test parameters change, the algorithm must change. Similarly, if the algorithm changes, the test protocol must change.

In yet other embodiments, the markers are captured and tested using non-SELDI formats. In one example, the sample is collected from the patient. The biomarkers are captured on a substrate using other known means, e.g., antibodies to the markers. The markers are detected using methods known in the art, e.g., optical methods and refractive index. Examples of optical methods include detection of fluorescence, e.g., ELISA. Examples of refractive index include surface plasmon resonance. The results for the markers are then subjected to an algorithm, which may or may not require artificial intelligence. The algorithm produces a diagnosis based upon the data received relating to each biomarker.

In any of the above methods, the data from the sample may be fed directly from the detection means into a computer containing the diagnostic algorithm. Alternatively, the data obtained can be fed manually, or via an automated means, into a separate computer that contains the diagnostic algorithm.

### VII. Diagnosis of Subject and Determination of Ovarian Cancer Survival Status

This panel of biomarkers comparing inter-alpha (globulin) inhibitor H4 (plasma Kallikrein-sensitive glycoprotein) (ITIH4), transferrin (TFR), and beta-2 microglobin (B2M), is useful in aiding in the determination of ovarian cancer status. First, the selected biomarkesr are measured in a subject sample using the methods described herein, e.g., capture on a SELDI biochip followed by detection by mass spectrometry. Then, the measurements is compared with a reference amount or control that allows for determination of the subject's prognosis. The test amounts as compared with the prognostic amount thus indicates ovarian cancer prognosis.

While individual biomarkers are useful diagnostic markers, it has been found that the particular combination of biomarkers provides herein provides surprisingly greater predictive value than single markers alone or other combinations of markers previously disclosed in the art. Specifically, the detection of this panel of markers in a sample increases the percentage of true positive and true negative diagnoses and would decrease the percentage of false positive or false negative diagnoses. Thus, methods of the present invention comprise the measurement of more than one biomarker.

The correlation may take into account the amount of the marker or markers in the sample compared to a control amount of the marker or markers (up or down regulation of the marker or markers) (e.g., in normal subjects in whom human cancer is undetectable). A control can be, e.g., the average or median amount of marker present in comparable samples of subjects in which their prognosis is known. The control amount is measured under the same or substantially similar experimental conditions as in measuring the test amount.

In certain embodiments of the methods of qualifying ovarian cancer status, the methods further comprise managing subject treatment based on the status. As aforesaid, such management describes the actions of the physician or clinician subsequent to determining ovarian cancer status. For example, if the result of the methods of the present invention is inconclusive or there is reason that confirmation of status is necessary, the physician may order more tests. Alternatively, if the status indicates that surgery is appropriate, the physician may schedule the patient for surgery. In other instances, the patient may receive chemotherapy either in lieu of, or in addition to, surgery. Likewise, if the result is negative, e.g., the status indicates late stage ovarian cancer or if the status is otherwise acute, no further action may be warranted. Furthermore, if the results show that treatment has been successful, no further management may be necessary.

The invention also provides for such methods where the biomarkers (or specific combination of biomarkers) are measured again after subject management. In these cases, the methods are used to monitor the status of the cancer, e.g., response to cancer treatment, remission of the disease or progression of the disease. Because of the ease of use of the methods and the lack of invasiveness of the methods, the methods can be repeated after each treatment the patient receives. This allows the physician to follow the effectiveness of the course of treatment. If the results show that the treatment is not effective, the course of treatment can be altered accordingly. This enables the physician to be flexible in the treatment options.

In another example, the methods for detecting markers can be used to assay for and to identify compounds that modulate expression of these markers in vivo or in vitro.

### VIII. Kits

In yet another aspect, the present invention provides kits for qualifying ovarian cancer status, e.g., for determining the prognosis of a subject, wherein the kits can be used to measure the markers of the present invention. For example, the kits can be used to measure the panel of markers described herein, which are useful in determining the prognosis of a subject with ovarian cancer. The kits can also be used to monitor the patient's response to a course of treatment, enabling the physician to modify the treatment based upon the results of the test. In another example, the kits can be used to identify compounds that modulate expression of one or more of the markers in in vitro or in vivo animal models for ovarian cancer.

The present invention therefore provides kits comprising (a) a capture reagent that binds the panel of three biomarkers; and (b) a container comprising at least one of the biomarkers. The capture reagents may also bind at least one known biomarker, Marker 4, e.g., CA125.

While the capture reagents can be any type of reagent, preferably the reagent is a SELDI probe. In certain kits of the present invention, the capture reagent comprises an MAC. In other embodiments, the reagent is an antibody.

Certain kits of the present invention further comprise a wash solution, or eluant, that selectively allows retention of the bound biomarkers to the capture reagents as compared with other biomarkers after washing. Alternatively, the kit may contain instructions for making a wash solution, wherein the combination of the adsorbent and the wash solution allows detection of the markers using gas phase ion spectrometry.

Preferably, the kit comprises written instructions for use of the kit for detection of the three biomarkers set forth herein and the instructions provide for contacting a test sample with the capture reagents and detecting the panel of biomarkers retained by the capture reagents. For example, the kit may have standard instructions informing a consumer how to wash the capture reagents (e.g., probes) after a sample of blood serum contacts the capture reagents. In another example, the kit may have instructions for pre-fractionating a sample to reduce complexity of proteins in the sample. In another example, the kit may have instructions for automating the fractionation or other processes.

Such kits can be prepared from the materials described above, and the previous discussion of these materials (e.g., probe substrates, capture reagents, adsorbents, washing solutions, etc.) is fully applicable to this section and will not be repeated.

In another embodiment, a kit comprises (a) antibodies that specifically bind to the panel of biomarkers; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a good or bad prognosis for a subject having ovarian cancer.

The invention also provides an article manufacture comprising at least one capture reagent bound to the panel of biomarkers provided herein. Examples of articles of manufacture of the present invention include, but are not limited to, ProteinChip® Arrays, probes, microtitre plates, beads, test tubes, microtubes, and any other solid phase onto which a capture reagent can be incorporated.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

### Example 1

### Proteomic Techniques Provide Insights into Human Ovarian Cancer Subjects Prognosis

Epithelial ovarian cancer (OC) is one of the leading causes of gynaecological cancer death worldwide. From the nationwide Danish Gynecologic Cancer Database (DGCD) it is known that on average 470 new OC cases and 140 Low Malignant Potential (LMP) ovarian tumors appear each year in Denmark [1]. From the DGCD it has been shown that the 3-year overall survival stage I-IV OC patients is 53%. For stage III OC patients the overall survival is 41%, much lower than the 3-year overall survival of 89% for stage I OC patients [1]. Because DGCD was initiated in 2005, only 3-year stage related survivals are available.

The relatively asymptomatic nature of early stage disease and the lack of adequate screening tests are the main reasons why more than 70% of cases present with late-stage disease (International Federation of Gynecology and Obstetrics (FIGO) stage III or stage IV). The 5-year overall survival for women diagnosed with late-stage disease is less than 20%, whereas the corresponding 5-year survival for women with early-stage disease (FIGO stage I and II) is approximately 90% [2, 3]. Recent studies have concluded that OC patients treated by gynecologic oncologists have better outcomes than patients treated by general gynecologists or general surgeons [4-7]. Therefore, the choice of debulking rate can be considered one prognostic factor.

The traditional clinicopathological variables of prognosis in OC, such as stage, histological grade, residual tumor and age, although highly useful, still have limitations in predicting the outcome of individual patients due to disease heterogeneity [8-11]. Therefore, additional and better factors indicative for overall and progression-free survival are needed.

A large number of new potential biological and cytotoxic treatments of OC have recently emerged. These new treatment modalities have resulted in an overwhelming interest in predictive and prognostic markers that can individualize OC treatment. Although many predictive factors have been found in OC, no reliable method for selecting patients for individualized treatments has been described so far. Clearly, the need for useful prognostic factors in order to optimize treatment of the patients diagnosed with OC has to be emphasized.

Proteomic approaches may provide new insights into biomarker discovery and application. Techniques such as surface-enhanced laser desorption/ionization time of flight-mass spectrometry (SELDI-TOF-MS, SELDI) have the potential to measure large number of proteins in a single sample [12]. Petricoin et al. [13] discovered patterns of proteins found in the blood of OC patients, and reported 100% sensitivity and 95% specificity for the investigated set of serum samples. Unfortunately, other OC data with the same level of sensitivity and specificity have not been reported [14]. Zhang et al. [15] used a multivariable model to combine apolipoprotein A1 (APOA1), transthyretin (cysteinylated form) (TT) and inter-alpha trypsin inhibitor IV (internal fragment) (ITIH4) values from 503 patients. Analysed in combination with serum CA125, the markers had a sensitivity of 74% and a specificity of 94% for detecting OC, which was an improvement over CA125 alone. A large-scale multi-centre study evaluated a set of seven biomarkers (ITIH4, TT, APOA1, transferrin (TrF), hepcidin (HEPC), connective-tissue activating protein 3 (CTAP3) and Serum Amyloid A1 (SAA), for the detection of OC. A total of 607 sera from five studies were analysed using SELDI-MS protocols optimized for the seven biomarkers. All seven biomarkers individually demonstrated statistically significant ability to discriminate for differentiation [16]. However, none of these references described the specific combination of inter-alpha (globulin) inhibitor H4 (plasma Kallikrein-sensitive glycoprotein), transferrin (TFR), and beta-2 microglobin (B2M) for determining overall survival or progression free survival, which is important in selecting an appropriate therapeutic regimen.

In addition to biomarker and therapeutic target discovery, proteomic techniques will likely provide insights into a patient's prognosis. Since patients with gross similarities in their disease burden do not share the same prognosis, differences in the tumor microenvironment likely contribute to their disparate outcomes. To clarify this, proteomics may provide additional information about potential confounding variables.

A study was undertaken of a prospective collection of women from the Danish Pelvic Mass study. All were candidates for surgery because of a suspicious pelvic mass. The aims were to determine if the serum proteomic biomarkers (APOA1, TT, HEPC, ITIH4, TrF, CTAP3 and B2M (beta-2 microglobulin)), alone or in combination, might be indicative of overall survival and/or progression-free survival for women diagnosed with OC. These seven biomarkers had not been previously evaluated in the above prognostic aspects of OC.

### Patient Collection

Between September 2004 and January 2008, 838 women admitted to the Gynecologic Clinic, Rigshospitalet, Denmark for surgery because of a pelvic mass, were enrolled into the “Pelvic Mass” study. Of these patients 150 were diagnosed with OC (Table 1). All eligible patients. ≧18 years with the suspicion of a pelvic mass were informed both in writing and verbally and were invited after written consent to participate in the study. Patients were examined with an abdominal and vaginal ultrasound and serum CA-125 was analysed. Exclusion criteria were pregnancy, previous cancer or borderline tumor, no understanding of information or cancellation of surgery because of no suspicion of pelvic disease after further examinations.

TABLE 1
Clinicopathological characteristics and biomarker levels in study subjects (N = 150).

| | APO1 | TT | HEPC | ITIH4 | B2M | CTAP3 | TrF |
|---|---|---|---|---|---|---|---|
| Peak intensities: | | | | | | | |
| Median | 6.97 | 4.25 | 7.36 | 3.32 | 2.90 | 2.48 | 1.15 |
| Range | 4.65-8.20 | 1.66-5.69 | 6.64-10.28 | 3.32-8.32 | 1.59-5.56 | 0.97-4.10 | 0.32-2.21 |
| Tumor stage (FIGO)* | 0.039 | 0.024 | 0.003 | 0.76 | 0.018 | 0.42 | 0.001 |
| Histological type of | 0.26 | 0.76 | 0.41 | 0.0005 | 0.0006 | 0.64 | 0.78 |
| tumor* | | | | | | | |
| Performance status* | 0.013 | 0.009 | 0.018 | 0.91 | <0.0001 | 0.017 | <0.0001 |
| Radicality of primary | 0.012 | 0.0004 | 0.0001 | 0.12 | <0.0001 | 0.027 | <0.0001 |
| surgery* | | | | | | | |

*p-value, Spearman Correlation Coefficients

A Risk Malignancy Index (RMI) was calculated based on the ultrasound score (U), the menopausal score (M), and value of serum CA125. Multilocularity (≧bilocular), solid areas, internal papilla, bilaterality, ascites, and extraovarian tumors scored one point each. A total of 2 or more points gave U=3; fewer than 2 points gave U=1. Postmenopausal status was defined as more than 1 year of amenorrhea or a previous hysterectomy and age ≧50. Premenopausal status scored M=1 and postmenopausal M=3. Serum CA125 was entered directly into the equation: RMI=U×M×CA125. If RMI was >200, positron emission tomography/computed tomography (PET/CT) was performed and the patient operated by a specialist in gynaecologic oncology. If RMI was ≦200 the patient could be operated by a general gynaecologist. In this study six patients had a RMI ≦200 and 144 patients had a RMI >200. All 150 patients were operated by a specialist in gynaecologic oncology. Surgery was performed through a midline incision with the intention of radical surgery. If necessary extensive surgery was performed in order to achieve macro-radical surgery and removal of all PET/CT positive tumors.

All tissue specimens were examined by a pathologist who specialized in gynecologic cancer. All patients were registered in DGCD, which is a compulsory research and quality on-line database. The FIGO stage distribution was 22 stage I patients, 14 stage II patients, 80 stage III patients and 34 stage IV patients. A total of 116 patients had serous adenocarcinama, 7 patients had mucinous adenocarcinoma and 27 patients had tumors of other histological types.

Furthermore, preoperative performance status for each patient was obtained from DGCD. Score 0: no signs of activity of disease (N=64), score 1: smaller signs of activity of disease (N=57), score 2: patient mobile for more than 50% of day time (N=27) and score 4: patient stay in bed, not mobile (N=2).

All cases in this study were traced in the Danish Central Population Register (CPR) and date of death, emigration up to January 8^{th} 2009, whichever came first, were registered. In addition, all women were linked to DGCD and information about treatment (surgery and chemotherapy) and cause of death was established. At the end of follow-up, a total of 62 OC patients had died from OC (median follow-up time: 11 months, range: 1-39) and 88 patients were still alive (median follow-up time: 40 months, range: 13-52).

After surgery 129 patients were treated with platinum-paclitaxel based chemotherapy, 1 patient was treated with Cyclophosphamide and 1 patient received Adriamycine treatment. A total of 19 patients did not receive chemotherapy (6 patients were FIGO stage IA highly differentiated, 1 patient had a stage IC, 7 patients were stage IIIC and 5 patients were stage IV). Twelve of the 19 patients were too sick to receive chemotherapy treatment.

Furthermore, information regarding progression was obtained from the oncological patient files. Standard WHO response criteria were used to verify response. In short, complete remission was defined as disappearance of all clinical symptoms and a serum CA125 level below 35 U/ml, —evaluated after completion of first-line chemotherapy, or if serum CA125 value had been higher than 35 U/ml preoperatively. Progression-free survival was calculated from the date of surgery to the date of documented disease progression (clinical, ultrasound, CT or PET/CT) and/or biochemical) or end of study, which was January 2008. The collection of progression data is a more time consuming process than collection of survival information from registries. Progression data is up to one year older than survival data. At the end of follow-up, a total of 80 OC patients had no clinical symptoms of progression (median progression free survival: 15 months, range: 1-41) and 70 patients had progression (median progression free survival: 4 months, range: 0-31).

The Danish Ethical Committee approved the protocol according to the rules used in International Conference on Harmonisation/Good Clinical Practice (ICH/GCP) recommendations and the Helsinki and Tokyo conventions (KF01-227/03 and KF01-143/04).

### Blood Sample Analysis

All blood samples were collected less than two weeks before surgery. The samples were sent by special car to the laboratory, centrifuged at 2000 g for 10 minutes at room temperature and fractionated into serum aliquots of approximately 0.5 ml and stored at −80° C. on the day of collection. Handling of blood samples from sampling to freezing was time stamped and recorded in the database in order to secure the time schedule. Aliquots used for the proteomic study were only been thawed for the actual study.

CA125

Serum CA125 was measured using a commercially available immuno assay, the CA125II assay (Kryptor reagents on the BRAHMS Kryptor, Immunodiagnostic systems, using the TRACE (Time Resolved Amplified Cryptate Emission) technology, based on non-radioactive transfer of energy. Intra-assay co-efficient of variation (CV) was 6.6% (n=60), whereas the inter-assay CV was 6.2% (n=10) at a control sample of 30 U/ml

Proteomic

The measurement of APOA1, TT, HEPC, ITIH4, B2M, CTAP3 and TrF could be accomplished in four assays, depending on the optimal ProteinChip array chemistry each analyte bound to. All sample and reference dilutions, and array processing steps were automated using a combination of commercially available automated workstations. Tecan MCA-150 Freedom EVO (Tecan, Durham, N.C.) and the BioMek 2000 (Beckman Coulter, Fullereton, Calif.) to prevent errors and maintain protocol consistency.

Data Collection and Analysis

After a final 30 minutes of drying, all arrays were processed in a ProteinChip SELDI System (Enterprise Edition, Bio-Rad Laboratories) using ProteinChip Data Management software v3.0. Data acquisition settings were optimized for the individual analytes and to provide the best performance. After all spectra were collected, data was archived and then imported into OvaCalc Software v3.1 (Vermillion Inc). This software package performed all calculations for the assay performance QC and the quantitative or semi-quantitative determinations of each of the seven analytes.

Statistical Analysis

Descriptive statistics are presented by the median and range. The Spearman rank correlation was used as a measure of association between quantitative variables. Tests for independence between categorical variables were done using the chi-square test and tests for location for continuous variables were done using the Wilcoxon rank sum test. Univariate survival probability curves for overall survival were performed on the entire study population (N=150) and on patients with residual tumor after surgery (N=92). The levels of the seven proteomic biomarkers and CA125 were scored by the log(base2) of the actual values. The impact of the proteomic prognostic index (xb-pro) on overall survival was estimated using the multivariable Cox proportional hazards model [17] removing proteomic variables which were not significant. The proteomic index was then constructed as the linear combination of the selected variables using the estimated regression coefficients. The chosen model was assessed using cross validation techniques [18]. The index values have been standardized by the mean value and standard deviation. Kaplan-Meier estimates of survival probabilities were calculated by grouping patients using the index tertiles as cutpoints. The equality of strata were tested using the log rank test.

The assumptions of proportionality and linearity were assessed using Schoenfeld and martingale residuals as well as graphical methods, the assumptions were not rejected. Multivariable Cox proportional hazard regression was done including the proteomic xb-pro index and adjusting for International Federation of Gynaecology and Obstetrics' (FIGO) stage (I, II, III and IV), residual tumor after primary surgery (yes/no), performance status (1, 2, 3, 4), age at diagnosis (linear), histological type of tumor (serous, mucinous, other types), serum CA125 levels and chemotherapy (yes/no). The results for each variable are presented by the hazard ratio (HR) and their 95% confidence intervals (95% CI). The same analysis was done for the endpoint PFS. P-values less than 5% were considered significant. All statistical calculations were done using a commercially available software package, SAS (v9.1, SAS Institute, Cary, N.C., USA).

### Example 2

### Description of the Seven Markers: APO1, TT, HEPC, ITIH4, B2M, CTAP3 and TrF

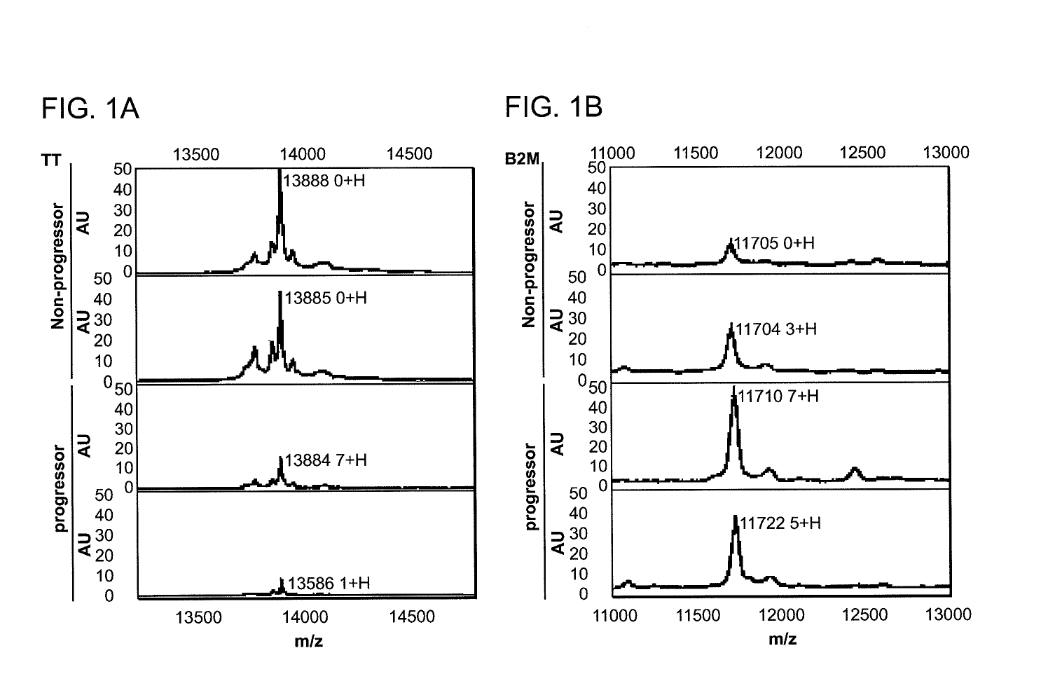

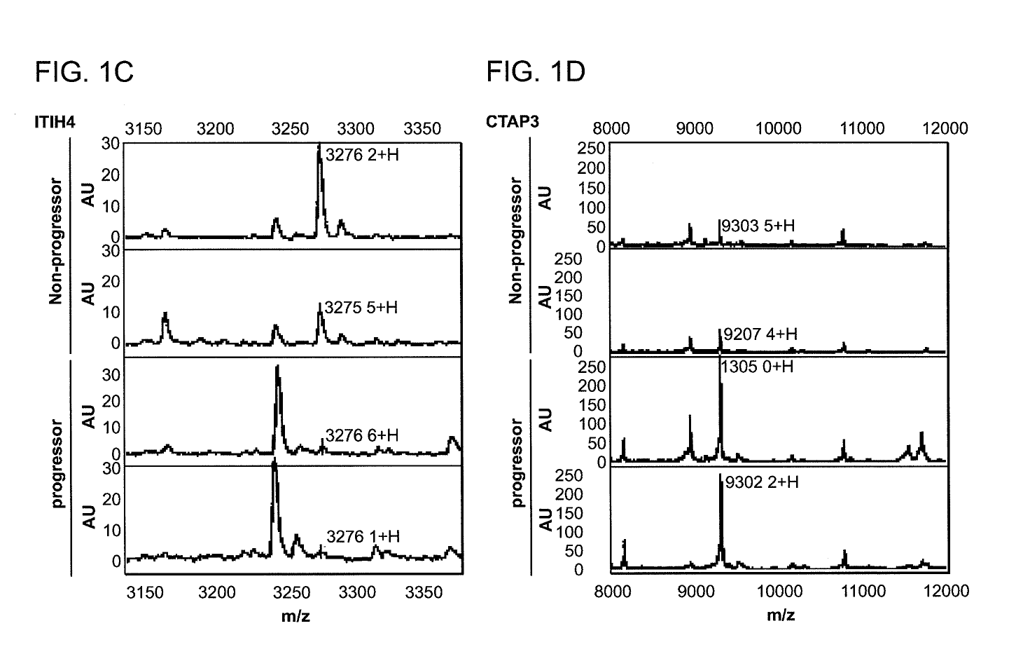

The patients included in the study were slightly older (65 years, range: 30-87) than the median age for Danish patients diagnosed with OC (60 year) [1]. Similarly with respect to stage and histology this study may reflect the group of women all treated at a University Hospital. For all OC patients the median peak intensity was 6.97 for APOA1 (range: 4.65-8.20), 4.25 for TT (range: 1.66-5.69), 7.36 for HEPC (range: 6.64-10.28), 3.32 for ITIH4 (range: 3.32-8.32), 2.90 for B2M (range: 1.53-5.56), 2.48 for CTAP3 (range: 0.97-4.10) and 1.15 for TrF (range: 0.32-2.21). The median serum CA125 level was 558.5 U/ml (range: 6-17275). Representative spectra from non-progressing OC patients and from progressing OC patients are shown in FIGS. 1A-1D.

A significant positive correlation was found between serum CA125 and peak intensities of HEPC (r=0.18, p=0.031), B2M (r=0.27, p=0.0009) and CTAP3 (r=0.26, p=0.001). A significant negative correlation was found between serum CA125 levels and peak intensities of APOA1 (r=−0.24, p=0.003), TT (r=−0.36, p<0.0001), TrF (r=−0.29, p=0.0003). All markers except ITIH4 correlated with each other (strongest correlation observed was 0.61 (absolute value)).

APOA1, TT, HEPC, B2M and TrF were all associated with FIGO stage (APOA1: p=0.039, TT: p=0.024, HEPC: p=0.003, B2M: p=0.018 and TrF: p=0.001, Wilcoxon rank sum test). The same markers in addition with CTAP3 were associated with performance status (APOA1: p=0.013, TT: p=0.009, HEPC: p=0.018, B2M: p<0.0001, TrF: p<0.0001, CTAP3: p=0.017, Wilcoxon rank sum test) and residual tumor after surgery (APOA1: p=0.012, TT: p=0.0004, HEPC: p=0.0001, B2M: p<0.0001, Trf: p<0.0001, CTAP3: p=0.027, Wilcoxon rank sum test). ITIH4 and B2M were associated with histological type of tumor (ITIH4: p=0.0005, B2M: p=0.0006, Wilcoxon rank sum test) (Table 1).

### Serum Proteomic Xb-Pro Index and Xb-Pfs Index for Overall and Progression-Free Survival.

Overall Survival—

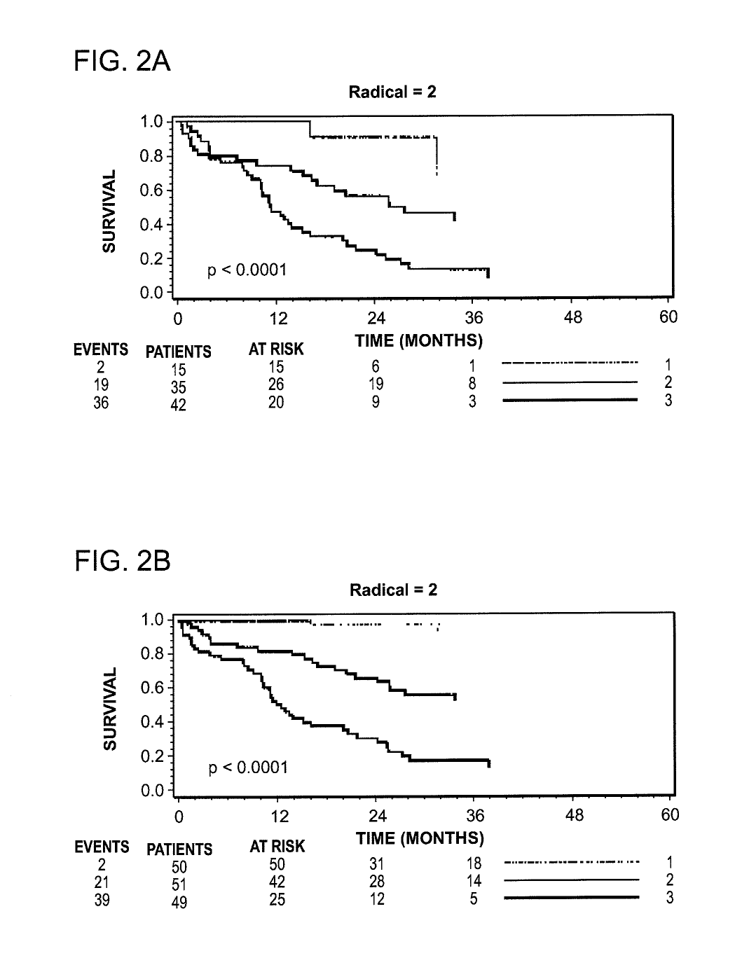

A total of 62 out of the 150 OC patients (41%) died during follow-up (2 in stage I, 4 in stage II, 37 in stage III and 19 in stage IV). Univariate analysis including all OC patients and the seven biomarkers as well as CA125 demonstrated a significant association with survival using the Cox proportional hazards model for APOA1, TI′, HEPC, B2M, CTAP3, TrF and CA125 whereas ITIH4 was not significant (Table 2). Kaplan-Meier curves demonstrating the association between xb-pro index and patients with residual tumor after surgery (N=92), divided into three groups using the first and second tertiles of the xb-pro index as cutpoints, are shown in FIG. 2A. Similarly, the association between the xb-pro index and all OC patients (N=150) is shown in FIG. 2B. For both patient groups a highly significant better survival was observed between patients with xb-pro index in the upper tertile compared with patients with lower xb-pro index values.

Performing a multivariable Cox survival analysis including all seven proteomic biomarkers in order to select a possible combination of proteomic markers forming a potential prognostic index, and with backwards reduction, the following biomarkers were included: ITIH4 (HR=0.67, 95% CI: 0.45-0.99, p=0.042), B2M (HR=3.07, 95% CI: 2.19-4.31, p<0.0001) and TrF (HR=0.13, 95% CI: 0.06-0.28, p<0.0001), whereas APOA1 (p=0.32), TT (p=0.41), HEPC (p=0.32), CTAP3 (p=0.18) were not found to have prognostic importance. B2M was the variable contributing most to the fit. Removing this variable from the analysis, it was found that only ITIH4 and TrF were included in the model. Removing TrF resulted in CTAP3 and HEPC being retained (p=0.003 and p=0.002 respectively). Both variables were moderately associated to TRF suggesting that these variables could replace TrF. Removing ITIH4 did not result in other variables being included. Cross validation of the model demonstrated that the estimates were robust (B2M: HR=3.09, (95% CI:2.70-3.54); TrF: HR=0.12, (95% CI:0.09-0.17); ITIH4: HR=0.66, (95% CI: 0.56-0.78)). None of the other variables contributed substantially to the model fit.

TABLE 2
Determinants of survival in 150 stage I-IV ovarian cancer patients.

| | | Ovarall survival (OS) | | | Progression-free survival (PFS) | | |
|---|---|---|---|---|---|---|---|
| Covariate | | HR (95% CI) | | p-value | HR (95% CI) | | p-value |
| FIGO Stage | III + IV vs I + II | 1.62 | (0.53-5.00) | 0.40 | 15.88 | (2.69-93) | 0.002 |
| Residual tumor | No vs yes | 8.24 | (2.50-27) | 0.0005 | 0.78 | (0.24-2.51) | 0.67 |
| after surgery | | | | | | | |
| Histological type | Mucinous vs serous | 2.18 | (0.49-9.63) | 0.17 | * | | 0.33 |
| | Other types vs serous | 1.70 | (0.94-3.07) | | 0.57 | (0.28-1.19) | |
| Chemotherapy | Yes vs no | 0.29 | (0.14-0.58) | 0.0006 | * | | |
| Performance status | >0 vs 0 | 1.38 | (0.71-2.65) | 0.34 | 4.20 | (1.73-10.15) | 0.0015 |
| Age | Per 10 years | 1.46 | (1.12-1.90) | 0.005 | 1.44 | (1.04-1.98) | 0.027 |
| Serum CA125 | Log base2 | 1.00 | (0.86-1.17) | 0.97 | 1.10 | (0.90-1.33) | 0.36 |
| Linear predictor | | 2.64 | (1.81-3.84) | <0.0001 | ** 1.85 | (1.17-2.92) | 0.009 |

* HR: Hazard Ratio
* Not included in analysis due to very low number of events.
** XB-PFS index

A multivariable Cox survival analysis including the xb-pro index (ITIH4, B2M and TrF) as a linear predictor adjusting for clinical covariates showed that xb-pro (p<0.0001, HR=2.50, 95% CI: 1.65-3.79, residual tumor after primary surgery (p=0.0005, HR=0.13, 95% CI: 0.04-0.41), age at diagnosis (p=0.01, HR=1.04, 95% CI: 1.01-1.07) and chemotherapy (p=0.0002, HR=0.22, 95% CI: 0.10-0.49) all are of independent prognostic value. FIGO stage, performance status, histological type of tumor and CA125 had no significant independent prognostic ability (Table 2). The type III tests show that the largest chi-square was the xb-pro index (18.49), chemotherapy (13.66), radicality after primary surgery (12.02), age at diagnosis (6.39), FIGO stage (4.96), histological type of tumor (4.23), performance status (3.02) and CA125 (0.56). The Cox survival analysis including the xb-pro index and the clinical covariates with independent prognostic value did not change the results.

Progression Free Survival

A Univariate Cox regression analyses confined to 120 OC patients (80 patients with no clinical symptoms of progression and 40 patients developing progression ≧1 month after primary surgery) presented the Xb-pro index as independent value of time to progression (p<0.0001, HR=2.19, 95% CI: 1.50-3.20).

A proteomic predictive index (xb-pfs) was constructed using the regression coefficients based on B2M (p=0.001, HR=2.82, 95% CI: 1.52-5.23) and CTAP3 (p=0.002, HR=4.09, 95% CI: 1.67-10.07). The other 5 serum proteomic markers were found of no value to predict progression-free survival. Cross validation suggested robust estimates of the linear predictor.

A multivariable Cox regression analysis including the proteomic xb-pfs index as a linear predictor adjusting for clinical covariates showed that xb-pfs (p=0.017, HR=1.84, 95% CI: 1.12-3.03). The results are shown in table 2. In a final Cox analysis, restricted to FIGO stage III patients, including the proteomic index xb-pfs, radicality of primary surgery, age and chemotherapy treatment showed proteomic xb-pfs of clinical independent predictive value when including radicality of primary surgery, age at diagnosis and treatment in the model (xb-pfs: p=0.008, HR=1.77, 95% CI: 1.17-2.70, radicality: p=0.02, HR=0.09, 95% CI: 0.01-0.64, age at diagnosis: p=0.04, HR=1.04, 95% CI: 1.00-1.08, chemotherapy: p=0.0006, HR=0.18, 95% CI: 0.07-0.48).

Protein expression profiling using proteomics techniques can be used to discover novel modified forms of proteins and to determine which combinations of proteins are most specifically associated with clinical conditions such as patient predictive value and prognosis. Because of its high mortality, OC has received much attention from proteomics analysis [13-16]. It is hoped that proteomics will allow the development of personalized patient therapy and monitoring of disease. Numerous markers have proven useful in individual studies. However, few have proven useful when applied to other different populations. This is one factor in determining the clinical relevance of candidate biomarkers. ApoA1, TT, and TrF are some of the biomarkers that have been successfully reproduced in other studies [15, 19-21].

So far no study has evaluated a specific combination or subset of the seven biomarkers delineated herein for either classification or for determining impact on overall and progression-free survival. A seven marker index has been evaluated for diagnostic use by Zhang et al., in which 6 of the markers are the same as the biomarkers investigated in this study. Zhang was able to differentiate between patients with OC and patients with benign tumors. However, they did not investigate the prognostic value of their biomarkers [16]. Unexpectedly, the three biomarkers ITIH4, B2M and TrF had significant independent prognostic value both when tested individually and in the xb-pro index. The 3 biomarkers found to be of high prognostic independent value as an index (xb-pro), have not earlier been investigated in OC in this respect, only as single biomarkers for differentiation of benign and malignant patients [22]. ITIH4 levels are enhanced in sera from OC patients compared to serum levels found in controls [23]. TT and B2M are reported of prognostic value in patients with Hodgkins disease and stage II colorectal cancer, respectively [24, 25]. This study indicates that the 3 biomarkers used in a proteomic prognostic index (xb-pro) may correlate with cancer. The index based on 3 biomarkers is even stronger than FIGO stage and performance status, which is quite unique for a biochemical index. Although none of these markers individually is specific for ovarian cancer, specificity is relatively unimportant in the narrow setting of determining prognosis. Indeed, it would be interesting to determine whether these indices might have prognostic value in other cancers.

Cross validation of the selection procedure demonstrated that B2M and TrF were included in more than 98% of the runs and ITIH4 was selected in more than 50% of the runs. Cross validation of the selected model comprising B2M, TrF and ITIH4 showed that the estimated hazard ratios were almost the same as those found in the final model suggesting robust estimates.

The biomarker B2M has been found predictive in patients with OC [26]. B2M is included in the proteomic xb-pro index and therefore the effect of this index on progression-free survival was analysed. The optimal proteomic index (xb-pfs) was composed of two biomarkers, B2M and CTAP3, with the strongest effect from B2M. Therefore, these findings support the earlier observation of B2M as a predictive independent marker of OC.

Further proteomic studies that elucidate differences in signaling cascades between these groups of women may enhance the clinician's ability to predict patients at highest risk for relapse at the time of diagnosis. This would promote rational treatment decisions that could prevent patients with early-stage disease from undergoing potentially harmful chemotherapy. In conclusion, seven serum biomarkers were evaluated alone and in combinations. A proteomic index (xb-pro) and its potential for predicting the outcome was investigated and a proteomic index (xb-pfs) and its potential for predicting progression-free survival for OC patients also investigated. The proteomic index had a very strong independent prognostic value for overall survival—even stronger than FIGO stage and B2M as reported earlier.

The panel of three biomarkers provides surprisingly accurate predictive results of survival independent of the stage of the cancer.

### Example 2

### Prognostic Panels of Biomarkers were Analyzed for Efficacy

Seven peaks were considered. All calculations have been done on the log scale (base 2). The chosen panel are: B2M_B, Trf_PR and ITIH4_D. These 3 have been validated as described. The p-values to include the others (TT_D, HEPC_D, APOA1_D and CTAP_D) are 0.66, 0.56, 0.33 and 0.35 (for OS). The following table presents univariable analyses of these peaks for Progression Free Survival (PFS) and overall survival (OS).

TABLE 3

| | PFS | | | OS | | |
|---|---|---|---|---|---|---|
| Covariate | p-value | HR | 95% CI | p-value | HR | 95% CI |
| B2M_B | 0.21 | 1.37 | 0.82-2.26 | <0.0001 | 2.73 | 1.84-4.04 |
| Trf_PR | 0.003 | 0.26 | 0.10-0.64 | <0.0001 | 0.14 | 0.06-0.35 |
| ITIH4_D | 0.30 | 0.78 | 0.49-1.24 | 0.054 | 0.55 | 0.30-1.01 |
| TT_D | 0.17 | 0.72 | 0.45-1.15 | 0.001 | 0.56 | 0.39-0.79 |
| HEPC_D | 0.19 | 1.33 | 0.87-2.04 | 0.006 | 1.58 | 1.14-2.18 |
| APO1_D | 0.18 | 0.68 | 0.39 | 0.010 | 0.51 | 0.30-0.85 |
| CTAP_D | 0.006 | 2.63 | 1.32-5.24 | 0.044 | 1.76 | 1.02-3.05 |

All peaks are significant for OS (note that ITIH4_d just over 0.05). In order to understand the multivariable analysis, the correlations between these variables are analyzed (Spearman rank correlations). See Table 4.

TABLE 4
(tc Spearman Correlations °\f C\12) Spearman Correlation Coefficients, N = 150
Prob > |r| under H0; Rho = 0

| | B2M_B | Trf_PR | ITIH4_D | TT_D | HEPC_D | APOA1_D | CTAP_D |
|---|---|---|---|---|---|---|---|
| B2M_B | 1.00000 | −0.18333 | 0.01806 | −0.29716 | 0.28793 | −0.26707 | 0.02930 |
| B2M B | | 0.0247 | 0.8264 | 0.0002 | 0.0004 | 0.0010 | 0.7219 |
| Trf_PR | −0.18333 | 1.00000 | 0.12736 | 0.52714 | −0.60877 | 0.49409 | −0.46741 |
| Trf PR | 0.0247 | | 0.1204 | <.0001 | <.0001 | <.0001 | <.0001 |
| ITIH4_D | 0.01806 | 0.12736 | 1.00000 | 0.02468 | 0.07251 | 0.15971 | −0.03057 |
| ITIH4 D | 0.8264 | 0.1204 | | 0.7643 | 0.3779 | 0.0509 | 0.7104 |
| TT_D | −0.29716 | 0.52714 | 0.02468 | 1.00000 | −0.43461 | 0.44210 | −0.24724 |
| TT D | 0.0002 | <.0001 | 0.7643 | | <.0001 | <.0001 | 0.0023 |
| HEPC_D | 0.28793 | −0.60877 | 0.07251 | −0.43461 | 1.00000 | −0.37010 | 0.17513 |
| HEPC D | 0.0004 | <.0001 | 0.3779 | <.0001 | | <.0001 | 0.0321 |
| APOA1_D | −0.26707 | 0.49409 | 0.15971 | 0.44210 | −0.37010 | 1.00000 | −0.34952 |
| APOA1 D | 0.0010 | <.0001 | 0.0509 | <.0001 | <.0001 | | <.0001 |
| CTAP_D | 0.02930 | −0.46741 | −0.03057 | −0.24724 | 0.17513 | −0.34952 | 1.00000 |
| CTAP D | 0.7219 | <.0001 | 0.7104 | 0.0023 | 0.0321 | <.0001 | |

Covariates which are highly correlated will result in only one being chosen in the multivariable analysis.

When the most significant peak is removed from the analysis (B2M_B, OS), this leads to only Trf_PR being retained (HR=0.14, 95% CI: 0.06-0.35, p<0.0001). In this model ITIH4_D is not included (p=0.12). The next step is to exclude Trf_PR with the following result: HEPC_D (HR=1.59, 95% CI:1.16-2.19, p=0.004) and ITIH4_D (HR=0.52, 0.27-0.98, p=0.044), now ITIH4_D is again in the model. This reflects the rather complicated covariance structure. The analysis is now done without HEPC_D, and this leads to TT_D being retained (HR=0.56, 95% CI:0.39-0.79, p=0.001). The next step (after removing TT_D) shows APOA1_D being included (HR=0.51, 95% CI:0.30-0.85, p=0.01). The final step includes CTAP_D (HR=1.76, 95% CI: 1.02-3.05, p=0.044, without ITIH4_D (p-value to include 0.06). These results demonstrate that the data are quite correlated leading to a predictive value for almost all variables, however the chosen panel is significantly better than the remaining. The roll of ITIH4_D is clearly associated with other peaks. This was also confirmed by the cross validation analyses.

Removing the chosen panel from the analysis leads to only TT_D being retained (HR=0.56, 95% CI:0.39-0.79, p=0.001). The chi-square value is 366.72 for the model fit, for the best model, the fit statistic is 329.44. The latter is substantially better than the first, indicating a much better fit for the best model.

Although almost all peaks contribute information on prognosis, the peaks B2M_B, Trf_PR and ITIH4_d describe the data considerably better than the peaks not chosen. Although ITIH4_D significantly improves the fit, its removal still results in a model substantially better than those not including B2M_B and Trf_PR. The results of the validation procedures demonstrate that the chosen panel (B2M_B, Trf_PR and ITH4_D) was robust in this dataset, i.e. none of the other covariates could reasonably replace these.

### Example 3

### A Panel Including B2M_B, Trf_PR and ITH4_D Had Prognostic Value

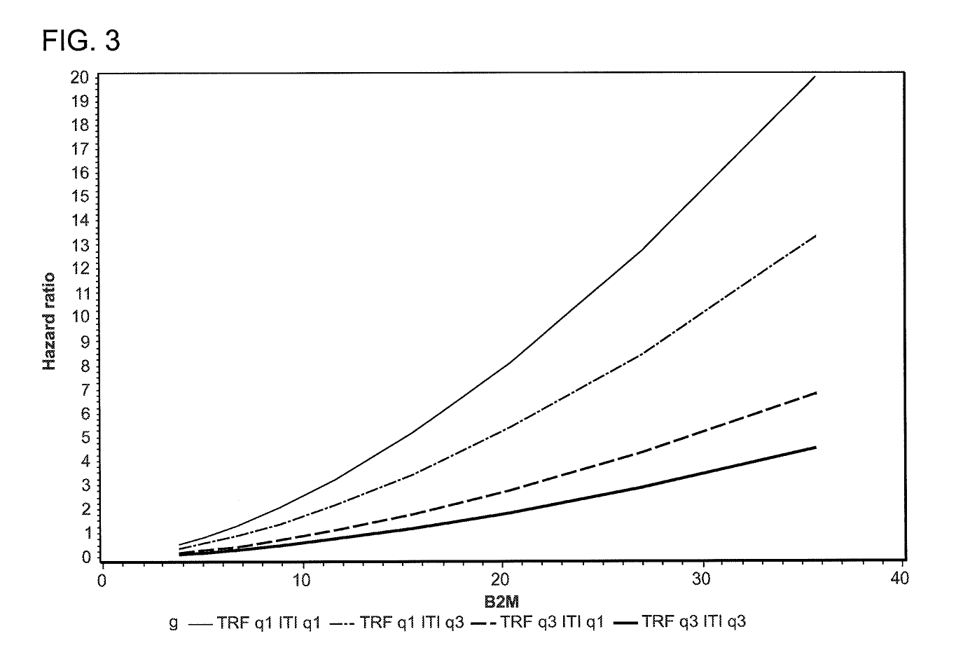

The 3 selected biomarkers are all statistically significant (p<0.05). The weakest covariate is ITIH4D. In a model including only B2M_B and TRF_PR, the hazard ratio for TRF_PR is 0.116 which is very similar to the result seen in the selected model (HR=0.126) whereas the HR for B2M_B increases to 3.074 with inclusion of ITIH4_D (versus 2.690 in the model without ITIH4_D). This suggests that the effect of B2M_B is mediated by the inclusion of ITIH4D, i.e. becomes stronger. The internal validation procedures suggested that B2M_B and TRF_PR are very robust estimates and that ITIH4 less so but still reasonably strong. See FIG. 3.

CA125 has been included in the univariable and multivariable analyses, please see the tables. CA125 is not significant in the multivariable setting.

### REFERENCES

1. Nielsen M L S, Høgdan C K. The Danish Gynecologic Cancer Database—A nationwide clinical database for ovarian cancer, endometrial cancer and cervical cancer, year 2006-2007. Loegeforeningens Forlag (ISSN 19017877) 2008, in press.
2. Lu K H, Patterson A P, Wang L et al. Selection of potential markers for epithelial ovarian cancer with gene expression arrays and recursive descent partition analysis. Clin Canc Res 2004, 10, 3291-3300.
3. Rapkiewicz A V, Espina V, Petricoin E F, Liotta L A. Biomarkers of ovarian tumours. Eur J Cancer 2004, 40, 2604-2612.
4. Soegaard Andersen E, Knudsen A, Svarrer T et al. The results of treatment of epithelial ovarian cancer after centralisation of primary surgery. Results from North Jutland, Denmark. Gynecol Oncol 2005, 99, 552-556.
5. Earle C C, Schrag D, Neville B A et al. Effect of surgeon specialty on processes of care and outcomes for ovarian cancer patients. J Natl Cancer Inst 2006, 98, 172-180.
6. Engelen M J, Kos H E, Willemse P H et al. Surgery by consultant gynecologic oncologists improves survival in patients with ovarian carcinoma. Cancer 2006, 106, 589-598.
7. Paulsen T, Kjaerheim K, Kaern J, Tretli S, Trope C. Improved short-term survival for advanced ovarian, tubal, and peritoneal cancer patients operated at teaching hospitals. Int J Gynecol Cancer 2006, 16 Suppl 1, 11-17.
8. van der Burg M E. Advanced ovarian cancer. Curr Treat Options Oncol 2001, 2, 109-118.
9. Nielsen J S, Jakobsen E, Hølund B, Bertelsen K, Jakobsen A. Prognostic significance of p53, Her-2, and EGFR overexpression in borderline and epithelial ovarian cancer. Int J Gynecol Cancer 2004, 14, 1086-1096.
10. Paramasivam S, Tripcony L, Crandon A et al. Prognostic importance of preoperative CA-125 in International Federation of Gynecology and Obstetrics stage I epithelial ovarian cancer: an Australian multicenter study. J Clin Oncol 2005, 23, 5938-5942.
11. Tingulstad S, Skjeldestad F E, Halvorsen T B, Hagen B. Survival and prognostic factors in patients with ovarian cancer. Obstet Gynecol 2003, 101, 885-891.
12. Britten R A, Hardy C, Vlahou A, Gregory B, Giri P S, Drake R. Identification of reproducible low mass SELDI protein profiles specific to cisplatin resistance in human ovarian cancer cells. Oncol Rep 2005, 14, 1323-1330.
13. Petricoin E F, Ardekani A M, Hitt B A et al. Use of proteomic patterns in serum to identify ovarian cancer. Lancet 2002, 359, 572-577.
14. Baggerly K A, Morris J S, Coombes K R. Reproducibility of SELDI-TOF protein patterns in serum: comparing datasets from different experiments. Bioinformatics 2004, 20, 777-785.
15. Zhang Z, Bast Jr R C, Yu Y et al. Three biomarkers identified from serum proteomic analysis for the detection of early stage ovarian cancer. Cancer Res 2004, 64, 5882-5890.
16. Zhang Z, Bast Jr R C, Vergote I et al. A large-scale multi-center independent validation study of a panel of seven biomarkers for the detection of ovarian cancer. J Clin Oncol 2006, 24, 5057.
17. Cox D R. Regression models and life tables. J Roy Stat Soc. B 1972, 34, 187-220.
18. Harrel F E J, Lee K I, Mark D B. Multivariate prognostic models: issues in developing models, evaluating assumptions and adequacy, and measuring and reducing errors. Stat Med 1996, 15, 361-387.
19. Rai A J, Zhang Z, Rosenzweig J et al. Proteomic approaches to tumor marker discovery. Arch Pathol Lab Med 2002, 126, 1518-1526.
20. Kozak K R, Su F, Whitelegge J P, Faull K, Reddy S, Farias-Eisner R. Characterization of serum biomarkers for detection of early stage ovarian cancer. Proteomics 2005, 5, 4589-4596.
21. Kozak K R, Amneus M W, Pusey S M et al. Identification of biomarkers for ovarian cancer using strong anion-exchange ProteinChips: potential use in diagnosis and prognosis. Proc Natl Acad Sci USA 2003, 100, 12343-12348.
22. Zhao Q, Duan W, Wu Y M, Qian X H, Deng X H. [Analysis of serum biomarkers of ovarian epithelial cancers based on 2-DE DIGE and MALDI TOF/TOF]. Zhonghua Zhong Liu Za Zhi 2008, 30, 754-758.
23. Mohamed E, Abdul-Rahman P S, Doustjalali S R et al. Lectin-based electrophoretic analysis of the expression of the 35 kDa inter-alpha-trypsin inhibitor heavy chain H4 fragment in sera of patients with five different malignancies. Electrophoresis 2008, 29, 2645-2650.
24. Hann H W, Lange B, Stahlhut M W, McGlynn K A. Prognostic importance of serum transferrin and ferritin in childhood Hodgkins disease. Cancer 1990, 15, 313-316.
25. Blum C, Graham A, Yousefzadeh M et al. The expression ratio of Map7/B2M is prognostic for survival in patients with stage II colon cancer. Int J Oncol 2008, 33, 579-584.
26. Hernadi Z, Molnar V, Juhasz B, Polka R, Margitai B. Der pradiktive Wert der Tumormarkerkombination CA-125 and Beta-2-Mikroglobulin bei Ovarialkarzinom. Zentralbl Gynakol 1992, 114, 6-9.

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A method of determining the prognosis of a subject having or suspected of having ovarian cancer, the method comprising comparing the concentration, expression, peak intensity or level of biomarkers transferrin (TFR) and beta-2 microglobin (B2M) or fragments thereof in a sample from the subject to the level present in a reference, wherein an increased level of said biomarkers relative to the reference is indicative of a poor prognosis.

2. (canceled)

3. The method of claim 1, further comprising comparing the level of connective-tissue activating protein 3 (CTAP3) or fragments thereof to a reference, wherein an increased level of said biomarker relative to the reference is indicative of a poor prognosis.

4. The method of claim 3, further comprising comparing the level of biomarkers CA125, hepcidin (HEPC), or fragments thereof in a sample from the subject to the level present in a reference, wherein an increased level of said biomarkers relative to the reference is indicative of a poor prognosis.

5. A method of determining the prognosis of a subject having or suspected of having ovarian cancer, the method comprising comparing the level of biomarkers apolipoprotein A1 (APOA1). transthyretin (TT), HEPC, B2M, CTAP3, TFR and CA125 or fragments thereof in a sample from the subject to the level present in a reference, wherein an increased level of said biomarkers relative to the reference is indicative of a poor prognosis.

6. The method of claim 1, further comprising comparing the level of one or more additional biomarkers to the level present in a reference, “wherein the additional biomarkers are selected from the group consisting of apolipoprotein A1, transthyretin, inter-alpha trypsin inhibitor IV, transferrin, hepcidin, connective-tissue activating protein 3, and Serum Amyloid A1 and beta-2 microglobin.

7. (canceled)

8. The method of any of claim 1, wherein the method further comprises considering one or more of the following factors in determining prognosis: radicality of primary surgery, age at diagnosis and treatment, FIGO stage, and histological type of tumor.

9. (canceled)

10. The method of claim 1, wherein the prognosis is predictive of overall survival or progression-free survival.

11. The method of claim 1, wherein failure to detect an increased level in one or more of said biomarkers is indicative of a good prognosis.

12. The method of claim 1 cla 9, wherein a subject's prognosis is used in selecting a therapeutic regimen.

13. The method of claim 12, wherein a poor prognosis indicates that the subject requires an aggressive therapeutic regimen and a good prognosis indicates that the subject requires a less aggressive therapeutic regimen.

14. The method of claim 13, wherein an aggressive therapeutic regimen includes neoadjuvant chemotherapy.

15. The method of claim 1 e wherein the overall survival or progression free survival is selected from the group consisting of one to two years survival post diagnosis; two to five years post diagnosis; and beyond five years post diagnosis.

16. A method of qualifying ovarian cancer status in a subject comprising:
(a) providing a subject sample of blood or a blood derivative;
(b) fractionating proteins in the sample on an anion exchange resin and collecting fractions that contain inter-alpha (globulin) inhibitor H4 (plasma Kallikrein-sensitive glycoprotein) (ITIH4), transferrin (TFR), and beta-2 microglobin (B2M).

17-20. (canceled)

21. The method of claim 1, wherein the sample is selected from the group consisting of ovarian tissue, lymph nodes, tissue biopsyₐ ovarian cyst fluid, ascites, pleural effusion, urine, blood, serum, and plasma.

22. (canceled)

23. A kit comprising:
(a) capture reagents that bind a biomarker of claim 5; and
(b) instructions for using the capture reagents to detect the biomarkers.

24-27. (canceled)

28. An article of manufacture comprising a panel of capture reagents that bind the panel of biomarkers of claim 1 or fragments of the respective biomarkers thereof.

29-34. (canceled)
